(19) United States
(12) Patent Application Publication
(10) Pub. No.: US 2008/0027075 A1
(43) Pub. Date: Jan. 31, 2008

(54) Quaternary Amino-Function Chalcones

(75) Inventor: Simon Feldbaek Nielsen, Herlev (DK)

Correspondence Address: EDWARDS ANGELL PALMER & DODGE LLP, P.O. BOX 55874, BOSTON, MA, 02205, US

(73) Assignee: Lica Pharmaceuticals A/S, Copenhagen (DK)

(21) Appl. No.: 10/577,614

(22) PCT Filed: Oct. 28, 2004
(86) PCT No.: PCT/DK04/00746

§ 371 Date: Apr. 20, 2007

(30) Foreign Application Priority Data
Oct. 31, 2003 (DK) PA 2003 01617

(51) Int. Cl. A61K 31/14; A61K 31/495; A61P 31/04; C07C 225/16; C07C 225/22; C07D 295/10

(52) U.S. Cl. 514/255.03; 514/643; 544/399; 564/286; 564/287

(57) ABSTRACT

The present invention provides novel quaternary amino-functional chalcone derivatives and analogues exhibiting interesting antibacterial activity. The compounds have the general formula (Y¹)ₘ—Ar¹(X¹)—C(=O)VAr²(X²)−(Y²)ₚ (I), wherein V designates —CH₂—CH₂—, —CH═CH— or —C≡C—; Ar¹ and Ar² independently are aryl and heteroaryl; m=0, 1, 2, p=0, 1, 2, m+p>0; each Y¹ and Y² independently is -Z—N⁺(R¹)(R²)R⁴ Q⁻ (A); —NR³-Z—N⁺(R¹)(R²)R⁴ Q⁻ (B), or —O-Z—N⁺(R¹)(R²)R⁴ Q⁻ (C), R¹, R², R³, R⁴, X¹, X² are substituents, and Q⁻ is an anion. The present invention also relates to the compounds for use as pharmaceutically active agents, in particular against bacterial infections (such as Gram-negative and Gram-positive bacteria, including antibiotic-sensitive or resistant strains), and in the medical treatment of bacterial infections in mammals.

### FIELD OF THE INVENTION

The present invention relates to a novel class of chalcone derivatives and analogues and to their use as pharmaceutically active agents, in particular against bacterial infections.

### BACKGROUND OF THE INVENTION

Chalcones, e.g., for use against bacterial infections are known from earlier patent applications assigned to the applicant, e.g. WO 93/17671 and WO 99/00114.

Moderate antibacterial activity has been reported for a limited number of chalcones in earlier publications e.g. Haraguchi, H. et al Phytochemistry 1998, 48, 125-129 and Hatano, T. et al Chem. Pharm. Bull (Tokyo) 2000, 48, 1286-92.

The spread of antimicrobial resistance determinants particular among nosocomial bacterial pathogens is an increasing problem. Such resistant pathogens include Staphylococcus aureus resistant to methicillin and thus to all β-lactam-antibiotics and Enterococci resistant to vancomycin (VRE). Such resistant bacteria pose a significant therapeutic challenge and bacterial strains resistant to all currently available antimicrobials are emerging. Furthermore, bacterial species intrinsically resistant to commonly employed antimicrobials are being recognized as important opportunistic pathogens in the setting of long-term immunocompromized patients. An example of this is Stenotrophomonas maltophilia which possesses a β-lactamase rendering the bacteria intrinsically resistant to carbapenems. As cross-resistance within a given class of antibiotics often occurs, the development of new classes of antibiotics is a neccisity to counter the emerging threat of bacterial resistance.

Thus, there is a need for chalcone derivatives and analogues with improved therapeutic activity against bacteria.

### BRIEF DESCRIPTION OF THE FIGURES

FIG. 1 illustrates the general synthetic scheme for the preparation of quaternary amino-functional chalcones from the corresponding amino-chalcones, where the aromatic rings are phenyl rings. R¹, R², and Z are as defined herein.

FIG. 2 illustrates a time-kill curve of D-003 against S. aureus E2371. Bacterial growth is inhibited at concentrations at or above the MIC. As CFU counts per ml decreases at concentrations of compound above the MIC, the compound is bactericidal. The reduction in CFU/ml is faster as the concentration of test compound increases above the MIC. This indicates that the bactericidal action of the compound is primarily dependent on the concentration of the test compound.

FIG. 3 illustrates a time-kill curve of D-005 against S. aureus ATCC29213. Bacterial growth is inhibited at concentrations at or above the MIC. As CFU counts per ml decreases at concentrations of compound above the MIC, the compound is bactericidal. The reduction in CFU/ml is faster as the concentration of test compound increases above the MIC. This indicates that the bactericidal action of the compound is primarily dependent on the concentration of the test compound.

FIG. 4 illustrates a time-kill curve of D-006 against S. aureus E2371. Bacterial growth is inhibited at concentrations at or above the MIC. As CFU counts per ml decreases at concentrations of compound above the MIC, the compound is bactericidal. The reduction in CFU/ml is faster as the concentration of test compound increases above the MIC. This indicates that the bactericidal action of the compound is primarily dependent on the concentration of the test compound.

FIG. 5 illustrates a time-kill curve of D-007 against S. aureus ATCC29213. Bacterial growth is inhibited at concentrations of test compound at or above the MIC. As CFU counts per ml decreases at concentrations of compound above the MIC, the compound is bactericidal. The rate of reduction of CFU/ml is not significantly affected by increasing concentrations of test compound. Thus, the bactericidal action of the compound is primarily dependent on incubation time.

FIG. 6 illustrates the general synthetic scheme for the preparation of amino-functional chalcones where the aromatic rings are phenyl rings. R¹, R², and Z are as defined herein.

FIG. 7 illustrates the synthesis of amino-dihydrochalcones. R¹, R², and Z are as defined herein.

FIG. 8 illustrates the general synthetic scheme for the preparation of diamino-functional chalcones where the aromatic rings are phenyl rings. R⁴=H (yielding the B ring) or CH₃ (yielding the A ring); R¹, R², R³ and Z are as defined herein.

FIG. 9 illustrates the scheme for the preparation of diamino (aminoacylamino)-functional chalcones where the aromatic rings are phenyl. R¹, R² and R³ are as defined herein, R₄=H or CH₃, and Y=(C(R^{H})₂)ₙ₋₁.

FIG. 10 illustrates the synthesis of diamino-dihydrochalcones. R¹, R², R³ and Z are as defined herein.

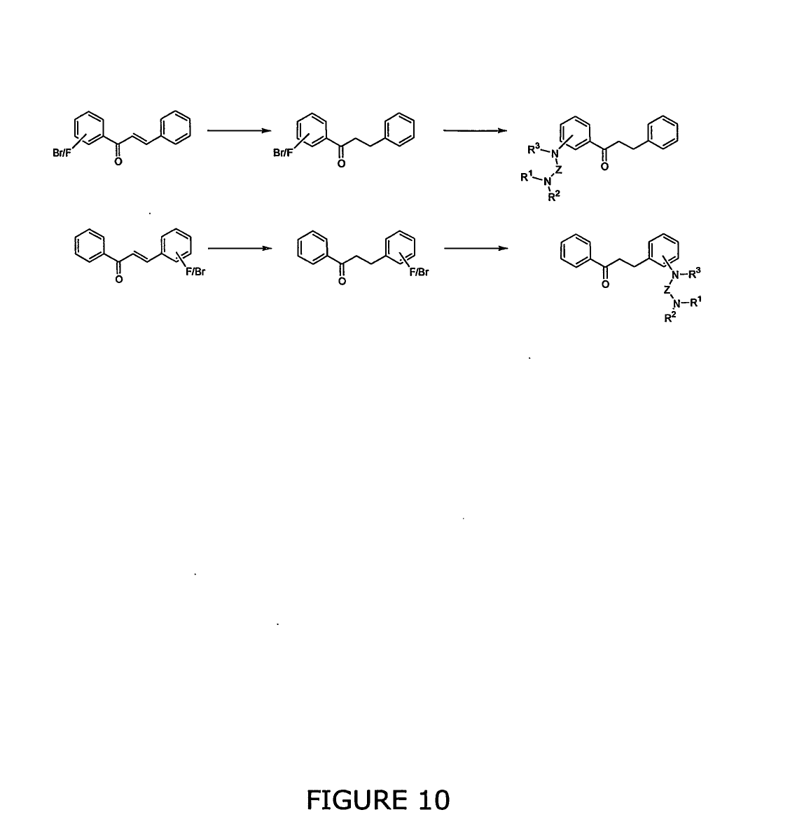

FIG. 11 illustrates the general synthetic scheme for the preparation of aminoalkoxy-functional chalcones where the aromatic rings are phenyl rings. R¹, R² and Z are as defined herein.

### DESCRIPTION OF THE INVENTION

The present inventors have found that quaternary amino-functional chalcones and analogues thereof exhibit an interesting antibacterial activity. This is highly surprising, as quaternary amino-functional compounds are not believed to be able to penetrate the bacterial membrane. The mechanism of action must, thus, be related to a direct effect on the membrane. This is supported by the time killing experiments that show an immediate and fast killing of bacteria.

Being permanently charged the compounds can only be given parenteral for systemic infections. As the compound cannot penetrate eukaryotic cells, the volume of distribution is very small (e.g. blood stream) meaning that very small amount of compound needs to be giving to achieve the therapeutic plasma concentration. The high plasma concentration will make the compounds ideally for the treatment of e.g. sepsis.

The compound is expected to be eliminated exclusively by the renal route. This means that high concentrations of the compound will be present in the urinary tract making the compounds especially attractive for the treatment of urinary tract infections.

Treatment of infections in the gastrointestinal system is also a very promising use if the compounds are giving by p.o. The compound cannot be absorbed resulting in a true local treatment of the infection.

Thus, in a first aspect, the present invention provides chalcone derivatives and analogues of the general formula I defined in claim 1, namely

(Y¹)ₘ—Ar¹(X¹)—C(=O)VAr²(X²)−(Y²)ₚ I

wherein

V designates —CH₂—CH₂—, —CH═CH— or —C≡C—;

Ar¹ and Ar² independently are selected from aryl and heteroaryl;

m is an integer selected from the group consisting of 0, 1, and 2,

p is an integer selected from the group consisting of 0, 1, and 2,

wherein the sum of m and p is at least 1;

each Y¹ and Y² independently represents a substituent selected from A, B, and C

-Z—N⁺(R¹)(R²)R⁴Q⁻, (A)

—NR³-Z—N⁺(R¹)(R²)R⁴Q⁻, and (B)

—O-Z—N⁺(R¹)(R²)R⁴Q⁻; (C)

wherein Z is a biradical —(C(R^{H})₂)ₙ—, wherein n is an integer in the range of 1-6 and each R^{H} is independently selected from hydrogen and C₁₋₆-alkyl, or wherein (R^{H})₂ is =O;

R¹, R² and R⁴ independently are selected from optionally substituted C₁₋₁₂-alkyl, optionally substituted C₂₋₁₂-alkenyl, optionally substituted C₄₋₁₂-alkadienyl, optionally substituted C₆₋₁₂-alkatrienyl, optionally substituted C₂₋₁₂-alkynyl, optionally substituted C₁₋₁₂-alkoxycarbonyl, optionally substituted C₁₋₁₂-alkylcarbonyl, optionally substituted aryl, optionally substituted aryloxycarbonyl, optionally substituted arylcarbonyl, optionally substituted heteroaryl, optionally substituted heteroaryloxycarbonyl, optionally substituted heteroarylcarbonyl, aminocarbonyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl; or R¹ and R²together with the nitrogen atom to which they are attached (—N(R¹)R²) form an optionally substituted nitrogen-containing heterocyclic ring;

R³ is selected from hydrogen, C₁₋₆-alkyl, and C₁₋₆-alkylcarbonyl, said alkyl and alkylcarbonyl optionally carrying substituent(s) selected from halogen, hydroxy, C₁₋₆-alkoxy, carboxy, C₁₋₆-alkoxycarbonyl, C₁₋₆-alkylcarbonyl, amino, mono- and di(C₁₋₆-alkyl)amino, and aryl optionally substituted 1-3 times with C₁₋₄-alkyl, C₁₋₄-alkoxy, nitro, cyano, amino or halogen; or R¹ and R³ together form a biradical Z^{*} which is as defined for Z;

Q is an anion;

X¹ and X² independently designate a substituent present 0-5 times on Ar¹ and Ar², respectively, each X¹ and X² being independently selected from the group consisting of optionally substituted C₁₋₁₂-alkyl, optionally substituted C₂₋₁₂-alkenyl, optionally substituted C₄₋₁₂-alkadienyl, optionally substituted C₆₋₁₂-alkatrienyl, optionally substituted C₂₋₁₂-alkynyl, hydroxy, optionally substituted C₁₋₁₂-alkoxy, optionally substituted C₂₋₁₂-alkenyloxy, carboxy, optionally substituted C₁₋₁₂-alkoxycarbonyl, optionally substituted C₁₋₁₂-alkylcarbonyl, formyl, C₁₋₆-alkylsulphonylamino, optionally substituted aryl, optionally substituted aryloxycarbonyl, optionally substituted aryloxy, optionally substituted arylcarbonyl, optionally substituted arylamino, arylsulphonylamino, optionally substituted heteroaryl, optionally substituted heteroaryloxycarbonyl, optionally substituted heteroaryloxy, optionally substituted heteroarylcarbonyl, optionally substituted heteroarylamino, heteroarylsulphonylamino, optionally substituted heterocyclyl, optionally substituted heterocyclyloxycarbonyl, optionally substituted heterocyclyloxy, optionally substituted heterocyclylcarbonyl, optionally substituted heterocyclylamino, heterocyclylsulphonylamino, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, amino-C₁₋₆-alkyl-carbonylamino, mono- and di(C₁₋₆-alkyl)amino-C₁₋₆-alkyl-carbonylamino, cyano, guanidino, carbamido, C₁₋₆-alkanoyloxy, C₁₋₆-alkylsulphonyl, C₁₋₆-alkylsulphinyl, C₁₋₆-alkylsulphonyloxy, aminosulfonyl, mono- and di(C₁₋₆-alkyl)aminosulfonyl, nitro, optionally substituted C₁₋₆-alkylthio, and halogen, where any nitrogen-bound C₁₋₆-alkyl is optionally substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino;

and salts thereof.

The group V is relevant with respect to the spatial orientation of the rings Ar¹ and Ar². Thus, the group V may be —CH₂—CH₂—, —CH═CH— or —C≡C. In a currently interesting embodiment thereof, V designates —CH═CH—.

In the context of the present invention, the expressions “chalcone derivative and analogues”, “chalcone derivatives/analogues” are to be assigned to the compounds of the above general formula I in that the overall structure namely Ar¹—C(=O)—C—C—Ar² resembles that of the chalcone structure. This being said, Ar¹ and Ar² are selected from aromatic rings and heteroaromatic rings. It is currently believed that particularly interesting compounds are those where at least one of Ar¹ and Ar², preferably both, are aryl, in particular phenyl. This being said, the inventors envisage that the functionality of the compounds may be substantially preserved (or even improved) when one or both of Ar¹ and Ar² are heteroaromatic rings.

In one embodiment, at least one of Ar¹ and Ar² is selected from thiazolyl, pyrrolyl, imidazolyl, pyrazolyl, pyridinyl, pyrimidinyl, pyrazinyl, pyridazinyl, thienyl, quinolyl, isoquinolyl, and indolyl.

In another embodiment, both of Ar¹ and Ar² are phenyl rings and Y¹ represents at least one quaternary amino-functional substituent, i.e. m is 1 or 2, and p is 0, 1 or 2.

In one embodiment, X¹ and X² independently designate 0-4, such as 0-3, e.g. 0-2, substituents, such optional substituents independently being selected from optionally substituted C₁₋₁₂-alkyl, hydroxy, optionally substituted C₁₋₁₂-alkoxy, optionally substituted C₂₋₁₂-alkenyloxy, carboxy, optionally substituted C₁₋₁₂-alkylcarbonyl, formyl, C₁₋₆-alkylsulphonylamino, optionally substituted aryl, optionally substituted aryloxycarbonyl, optionally substituted aryloxy, optionally substituted arylcarbonyl, optionally substituted arylamino, arylsulphonylamino, optionally substituted heteroaryl, optionally substituted heteroarylamino, optionally substituted heteroarylcarbonyl, optionally substituted heteroaryloxy, heteroarylsulphonylamino, optionally substituted heterocyclyl, optionally substituted heterocyclyloxy, optionally substituted heterocyclylamino, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, amino-C₁₋₆-alkyl-carbonylamino, mono- and di(C₁₋₆-alkyl)amino-C₁₋₆-alkylcarbonylamino, guanidino, carbamido, C₁₋₆-alkylsulphonyl, C₁₋₆-alkylsulphinyl, C₁₋₆-alkylsulphonyloxy, optionally substituted C₁₋₆-alkylthio, aminosulfonyl, mono- and di(C₁₋₆-alkyl)aminosulfonyl, and halogen, where any nitrogen-bound C₁₋₆-alkyl may be substituted with a substituent selected from the group consisting of hydroxy, C₁₋₆-alkoxy, and halogen.

In a more particular embodiment, X¹ and X² independently designate 0-3, e.g. 0-2, substituents, such optional substituents independently being selected from optionally substituted C₁₋₆-alkyl, hydroxy, optionally substituted C₁₋₆-alkoxy, carboxy, optionally substituted C₁₋₆-alkylcarbonyl, C₁₋₆-alkylsulphonylamino, optionally substituted aryl, optionally substituted aryloxy, optionally substituted arylamino, arylsulphonylamino, optionally substituted heteroaryl, optionally substituted heteroarylamino, heteroarylsulphonylamino, amino, mono- and di(C₁₋₆-alkyl)amino, carbamoyl, C₁₋₆-alkylcarbonylamino, guanidino, carbamido, optionally substituted C₁₋₆-alkylthio, optionally substituted heterocyclyl, optionally substituted heterocyclyloxy, optionally substituted heterocyclylamino and halogen, where any nitrogen-bound C₁₋₆-alkyl may be substituted with a substituent selected from the group consisting of hydroxy, C₁₋₆-alkoxy, and halogen.

In an even more particular further embodiment, X² represents at least one substituent selected from C₁₋₆-allyl, C₁₋₆-alkoxy, C₁₋₆-alkylcarbonyl, optionally substituted aryl, optionally substituted aryloxy, optionally substituted arylamino, optionally substituted heteroaryl, optionally substituted heteroarylamino, mono- and di(C₁₋₆-alkyl)amino, C₁₋₆-alkylcarbonylamino, optionally substituted C₁₋₆-alkylthio, optionally substituted heterocyclyl, optionally substituted heterocyclyloxy, optionally substituted heterocyclylamino and halogen.

In a yet further embodiment, X² represents at least one substituent selected from C₁₋₆-alkyl, C₁₋₆-alkoxy, optionally substituted aryl, optionally substituted aryloxy, optionally substituted heteroaryl, optionally substituted heteroarylamino, mono- and di(C₁₋₆-alkyl)amino, optionally substituted heterocyclyl and halogen.

In a yet even further embodiment, X² represents at least two halogen atoms.

The substituents Y¹ and Y² play an important role for the biological effect of the compounds and independently represent a substituent selected from A, B, and C

-Z—N⁺(R¹)(R²)R⁴Q⁻, (A)

—NR³-Z—N⁺(R¹)(R²)R⁴Q⁻, and (B)

—O-Z—N⁺(R¹)(R²)R⁴Q⁻; (C)

The Z group represents the biradical positioned between the ring (i.e. the aromatic or heteroaromatic ring, cf. Ar¹, Ar²) and the amino functionality. This group Z is typically a biradical —(C(R^{H})₂)ₙ—, wherein n is an integer in the range of 1-6, preferably 1-4, such as 1-3, or 2-3, and wherein each R^{H} is independently selected from hydrogen and C₁₋₆-alkyl, or two R^{H} on the same carbon atom may designate =O. A particular example of Z is —(CH₂)ₙ— wherein n is 1-4, such as 1-3, or 2-3.

In one embodiment, R¹, R² and R⁴ independently are selected from optionally substituted C₁₋₁₂-alkyl, optionally substituted C₂₋₁₂-alkenyl, optionally substituted C₂₋₁₂-alkynyl, optionally substituted C₁₋₁₂-alkylcarbonyl, arylcarbonyl, heteroarylcarbonyl, aminocarbonyl, mono- and di(C₁₋₆-alkyl)aminocarbonyl, amino-C₁₋₆-alkyl-aminocarbonyl, and mono- and di(C₁₋₆-alkyl)amino-C₁₋₆-alkyl-aminocarbonyl.

In a more particular embodiment, R¹, R² and R⁴ independently are selected from optionally substituted C₁₋₆-alkyl, optionally substituted C₁₋₆-alkylcarbonyl, heteroarylcarbonyl, aminocarbonyl, mono- and di(C₁₋₆-alkyl)aminocarbonyl, amino-C₁₋₆-alkyl-aminocarbonyl, and mono- and di(C₁₋₆-alkyl)amino-C₁₋₆-alkyl-aminocarbonyl.

In another embodiment, R¹, R² and R⁴ independently are selected from optionally substituted C₁₋₆-alkyl, optionally substituted C₂₋₆-alkenyl, optionally substituted aryl, and optionally substituted heteroaryl; or R¹ and R² together with the nitrogen atom to which they are attached (—N(R¹)R²) form an optionally substituted nitrogen-containing heterocyclic ring.

R³ is preferably selected from hydrogen and methyl, in particular methyl.

Thus, in a particular embodiment, one of Y¹ and Y² represents a substituent of the formula A

—CH₂—N⁺(R¹)(R²)R⁴Q⁻ (A)

wherein R¹, R² and R⁴ are independently C₁₋₆-alkyl. In particular at least Y¹ represents a substituent of the formula —CH₂—N⁺(R¹)(R²)R⁴ Q⁻.

In another particular embodiment, one of Y¹ and Y² represents a substituent of the formula B

—NR³—(CH₂)₂₋₃—N⁺(R¹)(R²)R⁴Q⁻ (B)

wherein R³ is selected from hydrogen and methyl, and R¹, R² and R⁴ are independently C₁₋₆-alkyl.

In a further particular embodiment, one of Y¹ and Y² represents a substituent of the formula C

—O—(CH₂)₂₋₃—N⁺(R¹)(R²)R⁴Q⁻ (C)

wherein R¹, R² and R⁴ are independently C₁₋₆-alkyl.

A particularly relevant variant of the before-mentioned particular embodiments is where V is —CH═CH—, and Ar¹ and Ar² both are phenyl.

Among the currently preferred specific compounds are:
(2-{3-[3-(2-Chloro-4-methoxy-phenyl)-3-oxo-propenyl]-3′,5′-dimethyl-biphenyl-4-yloxy}-ethyl)-trimethyl-ammonium, iodide;
(2-{3-[3-(4-Amino-phenyl)-3-oxo-propenyl]-3′,5′-dimethyl-biphenyl-4-yloxy}-ethyl)-trimethyl-ammonium, iodide;
(2-{3-[3-(2-Amino-phenyl)-3-oxo-propenyl]-3′,5′-dimethyl-biphenyl-4-yloxy}-ethyl)-trimethyl-ammonium, iodide;
4-{3-[3-(2-Fluoro-4-methoxy-phenyl)-3-oxo-propenyl]-2′-methoxy-biphenyl-4-yl}-1,1-dimethyl-piperazin-1-ium, iodide;
{3-[3-(4-Dibutylamino-phenyl)-acryloyl]-benzyl}-trimethyl-ammonium, iodide;
3-[4-(2-Trimethylammonium-ethoxy)-biphenyl-3-yl]-1-(3-trimethylammonium-phenyl)-propenone, di-iodide; and
3-[4-(2-trimethylammonium-ethoxy)-3′,5′-dimethyl-biphenyl-3-yl]-1-(2-trimethylammonium-4-methoxy-phenyl)-propenone, di-iodide.

It should be understood that the corresponding compounds where the anion, Q, is other than iodide are equally preferred.

In one preferred embodiment, m is 1 and p is 0. In another preferred embodiment m is 0 and p is 1. In a further interesting embodiment, m and p are both 1.

In a further typical embodiment, where Ar¹ and Ar² are both phenyl, V is —CH═CH—, Z is CH₂, R¹ and R² are methyl or together form a morpholino group, and one of m and p is 2 while the other of m and p is 0.

In a still further embodiment, the compound of the general formula I comprises at least 4 substituents in total.

In still further embodiment, the compound of the general formula I comprises at least two substituents in each ring Ar¹ and Ar².

As is evident from the formulae defined herein and the definitions associated therewith, certain compounds of the present invention are chiral. Moreover, the presence of certain cyclic fragments or multiple stereogenic atoms provides for the existence of diastereomeric forms of some of the compounds. The invention is intended to include all stereoisomers, including optical isomers, and mixtures thereof, as well as pure, partially enriched, or, where relevant, racemic forms. Thus, the above-mentioned compounds may be in the form of E- or Z-stereoisomers, or mixtures of such isomers. The E-isomers are generally preferred.

Embodiments where V is —CH═CH— may comprise E- and Z-stereoisomers, or mixtures of such isomers, which may exist in a dynamic equilibrium is solution. The E-isomers are generally preferred.

### Definitions

In the present context, the term “bacteriocidal” is intended to describe an antimicrobial activity of a test compound, characterized by the reduction of viable bacteria (bacterial kill) during incubation with the test compound, as evidenced in the killing curve determination by a reduction of colony forming units (CFU) during incubation time.

In the present context, the term “C₁₋₁₂-alkyl is intended to mean a linear, cyclic or branched hydrocarbon group having 1 to 12 carbon atoms, such as methyl, ethyl, propyl, iso-propyl, cyclopropyl, butyl, tert-butyl, iso-butyl, cyclobutyl, pentyl, cyclopentyl, hexyl, cyclohexyl, etc. Analogously, the term “C₁₋₆-alkly” is intended to mean a linear, cyclic or branched hydrocarbon group having 1 to 6 carbon atoms, such as methyl, ethyl, propyl, iso-propyl, pentyl, cyclopentyl, hexyl, cyclohexyl, and the term “C₁₋₄-alkyl” is intended to cover linear, cyclic or branched hydrocarbon groups having 1 to 4 carbon atoms, e.g. methyl, ethyl, propyl, iso-propyl, cyclopropyl, butyl, iso-butyl, tert-butyl, cyclobutyl.

Whenever the term “C₁₋₁₂-alkyl” is used herein, it should be understood that a particularly interesting embodiment thereof is “C₁₋₆-alkyl”.

Similarly, the terms “C₂₋₁₂-alkenyl”, C₄₋₁₂-alkadienyl”, and “C₆₋₁₂-alkatrienyl” are intended to cover linear, cyclic or branched hydrocarbon groups having 2 to 12, 4 to 12, and 6 to 12, carbon atoms, respectively, and comprising one, two, and three unsaturated bonds, respectively. Examples of alkenyl groups are vinyl, allyl, butenyl, pentenyl, hexenyl, heptenyl, octenyl, heptadecaenyl. Examples of alkadienyl groups are butadienyl, pentadienyl, hexadienyl, heptadienyl, heptadecadienyl. Examples of alkatrienyl groups are hexatrienyl, heptatrienyl, octatrienyl, and heptadecatrienyl. Preferred examples of alkenyl are vinyl, allyl, butenyl, especially allyl.

Similarly, the term “C₂₋₁₂-alkynyl” is intended to mean a linear or branched hydrocarbon group having 2 to 12 carbon atoms and comprising a triple bond. Examples hereof are ethynyl, propynyl, butynyl, octynyl, and dodecaynyl.

Whenever the terms “C₂₋₁₂-alkenyl”, “C₄₋₁₂-alkadienyl”, “C₆₋₁₂-alkatrienyl”, and “C₂₋₁₂-alkynyl” are used herein, it should be understood that a particularly interesting embodiment thereof are the variants having up to six carbon atoms.

In the present context, i.e. in connection with the terms “alkyl”, “alkenyl”, “alkadienyl”, “alkatrienyl”, and “alkynyl”, the term “optionally substituted” is intended to mean that the group in question may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxycarbonyl, aryloxy, arylamino, arylcarbonyl, heteroaryl, heteroarylamino, heteroaryloxycarbonyl, heteroaryloxy, heteroarylcarbonyl, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, halogen, where any aryl and heteroaryl may be substituted as specifically described below for “optionally substituted aryl and heteroaryl”, and any alkyl, alkoxy, and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Preferably, the substituents are selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkylcarbonyl, formyl, aryl, aryloxy, arylamino, arylcarbonyl, heteroaryl, heteroarylamino, heteroaryloxy, heteroarylcarbonyl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, guanidino, carbamido, C₁₋₆-alkyl-sulphonyl-amino, C₁₋₆-alkyl-sulphonyl, C₁₋₆-alkyl-sulphinyl, C₁₋₆-alkylthio, halogen, where any aryl and heteroaryl may be substituted as specifically described below for “optionally substituted aryl and heteroaryl”.

Especially preferred examples are hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, and guanidino.

The terms “optionally substituted C₁₋₂-alkoxy” and “optionally substituted C₁₋₆-alkoxy” are intended to mean that the alkoxy groups may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxycarbonyl, aryloxy, arylcarbonyl, heteroaryl, heteroaryloxycarbonyl, heteroaryloxy, heteroarylcarbonyl, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, halogen, where any aryl and heteroaryl may be substituted as specifically described below for “optionally substituted aryl and heteroaryl.

Especially preferred examples of “optionally substituted C₁₋₁₂-alkoxy” and “optionally substituted C₁₋₆-alkoxy” groups are unsubstituted such groups as well as those carrying one or two substituents selected from hydroxy, C₁₋₆-alkyl, C₁₋₆-alkoxy, C₂₋₆-alkenyloxy, carboxy, halogen, or C₁₋₆-alkylthio.

“Halogen” includes fluoro, chloro, bromo, and iodo.

In the present context, the term “aryl” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The term “heteroaryl” is intended to mean a fully or partially aromatic carbocyclic ring or ring system where one or more of the carbon atoms have been replaced with heteroatoms, e.g. nitrogen (=N— or —NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furyl, thienyl, quinolyl, benzothiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular pyrrolyl, imidazolyl, pyridinyl, pyrimidinyl, thienyl, quinolyl, tetrazolyl, and isoquinolyl.

The term “heterocyclyl” is intended to mean a non-aromatic carbocyclic ring or ring system where one or more of the carbon atoms have been replaced with heteroatoms, e.g. nitrogen (=N— or —NH—), sulphur, and/or oxygen atoms. Examples of such heterocyclyl groups are imidazolidine, piperazine, hexahydropyridazine, hexahydropyrimidine, diazepane, diazocane, pyrrolidine, piperidine, azepane, azocane, aziridine, azirine, azetidine, pyroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular imidazoildine, piperazine, hexahydropyridazine, hexahydropyrimidine, diazepane, pyrrolidine, piperidine, azepane, oxazinane (morpholine), and thiazinane.

In the present context, i.e. in connection with the terms “aryl”, “heteroaryl”, and heterocyclyl, the term “optionally substituted” is intended to mean that the group in question may be substituted one or several times, preferably 1-5 times, in particular 1-3 times) with group(s) selected from hydroxy (which when present in an enol system may be represented in the tautomeric keto form), C₁₋₆-alkyl, C₁₋₆-alkoxy, C₂₋₆-alkenyloxy, oxo (which may be represented in the tautomeric enol form), carboxy, C₁₋₆-alkoxycarbonyl, C₁₋₆-alkylcarbonyl, formyl, aryl, aryloxy, arylamino, aryloxycarbonyl, arylcarbonyl, heteroaryl, heteroarylamino, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, nitro, sulphanyl, amino, amino-sulfonyl, mono- and di(C₁₋₆-alkyl)amino-sulfonyl, dihalogen-C₁₋₄-alkyl, trihalogen-C₁₋₄-alkyl, halogen, where aryl and heteroaryl representing substituents may be substituted 1-3 times with C₁₋₄-alkyl, C₁₋₄-alkoxy, nitro, cyano, amino or halogen, and any alkyl, alkoxy, and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Preferably, the substituents are selected from hydroxy, C₁₋₆-alkyl, C₁₋₆-alkoxy, oxo (which may be represented in the tautomeric enol form), carboxy, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, C₁₋₆-alkylcarbonylamino, guanidino, carbamido, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, sulphanyl, amino, amino-sulfonyl, mono- and di(C₁₋₆-alkyl)amino-sulfonyl or halogen, where any alkyl, alkoxy and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino. Especially preferred examples are C₁₋₆-alkyl, C₁₋₆-alkoxy, amino, mono- and di(C₁₋₆-alkyl)amino, sulphanyl, carboxy or halogen, where any alkyl, alkoxy and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

In the present context the term “nitrogen-containing heterocyclic ring” is intended to mean heterocyclic ring or ring system in which at least one nitrogen atom is present. Such a nitrogen is, with reference to the general formula I (substituents A, B, and C), carrying the substituents R₁ and R₂. The “nitrogen-containing heterocyclic ring” may further comprise additional heteroatoms, e.g. nitrogen (=N— or —N—), sulphur, and/or oxygen atoms. Examples of such rings are aromatic rings such as pyridine, pyridazine, pyrimidine, pyrazine, triazine, thiophene, oxazole, isoxazole, thiazole, isothiazole, pyrrole, imidazole, pyrazole, tetrazole, quinoline, benzothiazole, benzotriazole, benzodiazole, benzoxozole, triazole, isoquinoline, indole, benzopyrazole, thiadiazole, and oxadiazole. The most interesting examples of aromatic rings are pyridine, pyridazine, pyrimidine, pyrazine, thiophene, tetrazole, oxazole, isoxazole, thiazole, isothiazole, pyrrole, imidazole, pyrazole, quinoline, triazole, isoquinoline, and indole, in particular pyridine, thiophene, imidazole, quinoline, isoquinoline, indole, and tetrazole.

Other examples of such rings are non-aromatic rings such as imidazolidine, piperazine, hexahydropyridazine, hexahydropyrimidine, diazepane, diazocane, pyrrolidine, piperidine, azepane, azocane, aziridine, azirine, azetidine, pyroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, and thiazetane. The most interesting examples of non-aromatic rings are imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular imidazolidine, piperazine, hexahydropyridazine, hexahydropyrimidine, diazepane, pyrrolidine, piperidine, azepane, oxazinane (morpholine), and thiazinane.

In the present context, i.e. in connection with the term “nitrogen-containing heterocyclic ring”, the term “optionally substituted” is intended to mean that the group in question may be substituted one or several times, preferably 1-5 times, in particular 1-3 times) with group(s) selected from the same substituents as defined above for “optionally substituted aryl”.

The anion, Q⁻, can be selected from halides, anions of organic acids, anions of mineral acids, etc. Examples hereof are chloride (Cl⁻), bromide (Br⁻), iodide (I⁻), acetate, lactate, fumerate, oxalate, sulphate, nitrate, etc.

This being said, it should furthermore be understood that the compounds defined herein include other salts (salts other than the quaternary amino salts having Q as the counter-ion) thereof, of which pharmaceutically acceptable salts are of course especially relevant for the therapeutic applications. Salts include acid addition salts and basic salts. Examples of acid addition salts are hydrochloride salts, fumarate, oxalate, etc. Examples of basic salts are salts where the (remaining) counter ion is selected from alkali metals, such as sodium and potassium, alkaline earth metals, such as calcium salts, potassium salts, and ammonium ions (⁺N(R¹)₄), where the R's independently designate optionally substituted C₁₋₆-alkyl, optionally substituted C₂₋₆-alkenyl, optionally substituted aryl, or optionally substituted heteroaryl). Pharmaceutically acceptable salts are, e.g., those described in Remington's—The Science and Practice of Pharmacy, 20th Ed. Alfonso R. Gennaro (Ed.), Uppincott, Williams & Wilkins; ISBN: 0683306472, 2000, and in Encyclopedia of Pharmaceutical Technology. However, generally preferred salt forming agents for application in the present invention are organic dicarboxylic acids such as oxalic, fumaric, and maleic acid, and the like.

### Preparation of Compounds

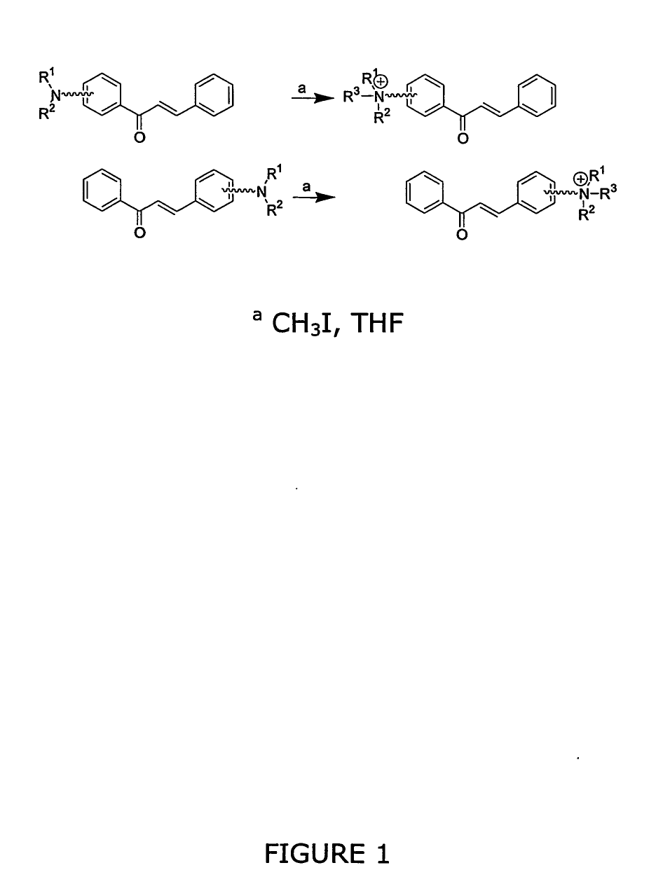

The quaternary compounds can be prepared by treatment of the corresponding amino-functional chalcone derivatives/analogues with an alkylating or acylating agent, e.g. an agent R⁴T, where R⁴ is as defined elsewhere herein and T is a leaving group (e.g. the anion Q) in a suitable solvent, e.g. as illustrated in FIG. 1 and described in the Examples section.

Typically, preparation of the quaternary amino-functional compounds proceeds involves treatment of a compound of the general formula II

(Y³)ₘ—Ar¹(X¹)—C(=O)VAr²(X²)−(Y⁴)ₚ II

wherein Ar¹, Ar², X¹, X², V, m and p are as defined elsewhere herein, and wherein each Y³ and Y⁴ independently represents a substituent selected from A′, B′, and C′

-Z—N(R¹)R², (A′)

—NR³-Z—N(R¹)R², and (B′)

—O-Z—N(R¹)R²; (C′)

wherein Z, R¹, R² and R³ are as defined elsewhere herein,
with an alkylating agent or an acylating agent, e.g. an agent R⁴T.

The reaction is typically conducted in a suitable solvent for the compound of general formula II at a temperature in the range of 0-100° C., e.g. 10-30° C. Reaction times are typically from 30 min to 24 hours. The solvent is typically an aprotic solvent, e.g. THF or the alkylating/acylation agent. The alkylating agent or the acylating agent is typically provided in an approximately equivalent ratio (e.g. about 1:1) for mono-alkylation/acylation or in large excess (e.g. up to 5:1 to 200:1, e.g. about 100:1) in the case of poly-alkylation/acylation.

The alkylating agent or acylating agent provides the group R⁴ and possibly also the counterion Q. Examples of alkylating agents are alkyl halides, e.g. alkyl iodides, such as methyl iodide, ethyl iodide. Examples of acylating agents are acyl halides, e.g. acetyl chloride and acid anhydrides e.g. acetic anhydride.

Alternatively, compounds of the general formula I may be prepared from other compounds of the general formula I by exchange of the anion Q, as will be appreciated by the person skilled in the art.

The corresponding amino-functional chalcone derivatives/analogues of the general formula II may be produced by methods known per se for the preparation of chalcones or methods which are analogous to such methods. Examples of excellent methods for preparing compounds of the 1,3-bis-aromatic-prop-2-enone or the 1,3-bis-aromatic-prop-2-ynone types are given in the following. Further examples of methods for the preparation of the compound used according to the present invention are described in WO 95/06628 and WO 93/17671 and in the references cited therein.

Compounds of the general formula II in which V is —CH═CH— can be prepared by reacting a ketone (an acetophenone in the case where Ar¹ is phenyl)

(Y³)ₘ—Ar₁(X¹)—C(=O)—CH₃

with an aldehyde (a benzaldehyde in the case where Ar² is phenyl)

HCO—Ar²(X²)−(Y⁴)ₚ

wherein Y³, Y⁴, Ar¹, Ar², X¹, X², m, and p refer to the definitions given elsewhere herein.

This reaction, which is a condensation reaction, is suitably carried out under acid or base catalysed conditions. A review of such processes may be found in Nielsen, A. T., Houlihahn, W. J., Org. React. 16, 1968, pp 1-444. In particular the method described by Wattanasin, S. and Murphy, S., Synthesis (1980) 647 has been found quite successful. The reaction may suitably be carried out in protic organic solvents, such as lower alcohols (e.g. methanol, ethanol, or tert-butanol), or lower carboxylic acids (formic, glacial acetic, or propionic acid), or in aprotic organic solvents such as ethers (e.g. tetrahydrofuran, dioxane, or diethyl ether), liquid amides (e.g. dimethylformamide or hexamethylphosphordiamide), dimethylsulfoxide, or hydrocarbons (e.g. toluene or benzene), or mixtures of such solvents. When carrying out the reaction under base catalysed conditions, the catalyst may be selected from sodium, lithium, potassium, barium, calcium, magnesium, aluminum, ammonium, or quaternary ammonium hydroxides, lower alkoxides (e.g. methoxides, ethoxides, tert-butoxides), carbonates, borates, oxides, hydrides, or amides of lower secondary amines (e.g. diisopropyl amides or methylphenyl amides). Primary aromatic amines such as aniline, free secondary amines such as dimethyl amine, diethyl amine, piperidine, or pyrrolidine as well as basic ion exchange resins may also be used.

Acid catalysts may be selected from hydrogen chloride, hydrogen bromide, hydrogen iodide, sulfuric acid, sulfonic acids (such as paratoluenesulfonic or methanesulfonic acid), lower carboxylic acids (such as formic, acetic or propionic acid), lower halogenated carboxylic acids (such as trifluoroacetic acid), Lewis acids (such as BF₃, POCl₃, PCl₃or FeCl₃), or acid ion exchange resins.

A drawback of the base catalysed condensation is the poor yield obtained if the aromatic ring in which the ketone or the aldehyde or both is substituted with one or more hydroxy groups. This drawback can be overcome by masking the phenolic group as described by T. Hidetsugu et al. in EP 0 370 461. Deprotection is easily performed by mineral acids such as hydrochloric acid.

The reaction is typically carried out at temperatures in the range of 0-100° C., e.g. at room temperature. Reaction times are typically from 30 min to 24 hours.

The starting materials for the synthesis (acetophenone and aldehyde), may be obtained from commercial sources or may be synthesised according to well-known methods.

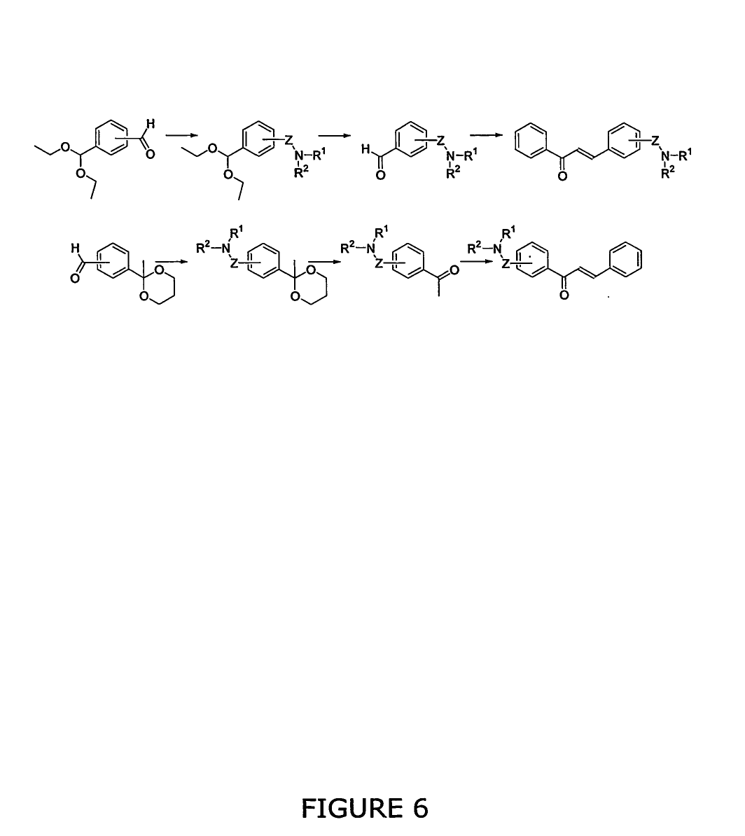

The alkyl- or dialkyl aminomethyl-acetophenones and -benzaldehydes were prepared by reductive amination using substituted benzaldehyde, amine and sodium triacetoxyborohydride. The alkyl- or dialkyl aminoalkyl-acetophenones and -benzaldehydes were prepared from the corresponding bromo-compounds using halogen/metal exchange (n-BuLi) and quenching with N,N-dimethylacetamide and dimethylformamide, respectively (FIG. 6).

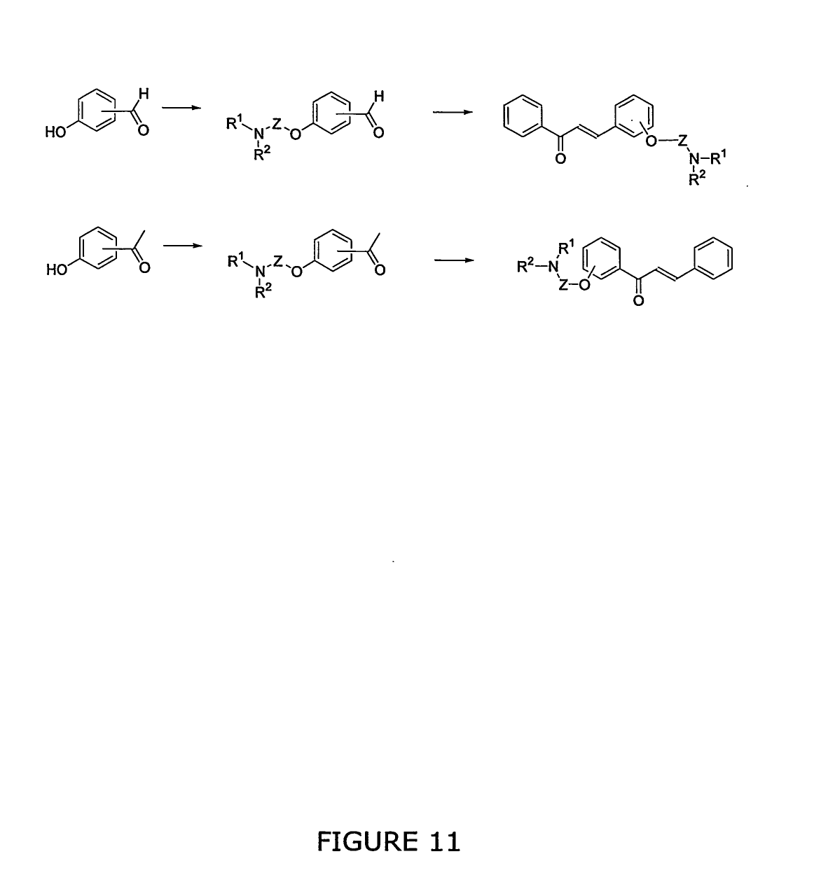

The aminoalkoxy-benzaldehydes and aminoalkoxy-acetophenones can be synthesized by alkylatlon of the corresponding hydroxy-benzaldehydes or hydroxy-acetophenones (FIG. 11). Alternatively, the aminoalkoxy-chalcone derivatives/analogues can be prepared by alkylation of the corresponding hydroxy-chalcone.

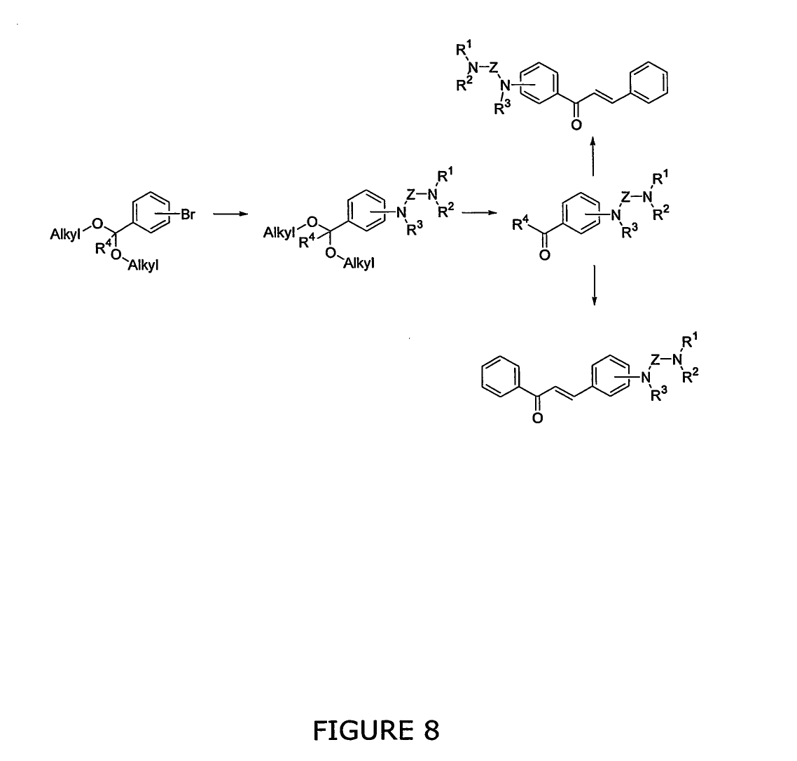

The diamino-benzaldehydes can be synthesized by Palladium catalysed reaction of bromo-benzaldehyde diethyl acetal and diamine followed by acidic work up. Alternatively, the 2-diamino-benzaldehydes can be prepared by nucleophilic aromatic substitution using 2-fluorobenzaldehyde and diamine. The diamino-acetophenones can be synthesized by Palladium catalysed reaction of bromoacetophenone ketal and diamine followed by acidic work up. Alternatively, the 2′-diamino-acetophenones can be synthesized by nucleophilic aromatic substitution using 2′-flouroacetophenone and diamine (FIG. 8).

Compounds of the general formula II in which V is —C≡C— may be prepared by reacting an activated derivative of a carboxylic acid of the general formula

(Y³)ₘ—(X¹Ar¹—COOH

with an ethyne derivative of the formula

H—C≡C—Ar²(X²)−(Y⁴)ₚ

wherein Ar¹, Ar², X¹, X², Y³, Y⁴, m, and p refer to the definitions given elsewhere herein.

Reactions of this type are described by Tohda, Y., Sonogashihara, K., Haghara, N., Synthesis 1977, pp 777-778. It is contemplated that the activated derivative of the carboxylic acid may be an activated ester, an anhydride or, preferably, an acid halogenide, in particular the acid chloride. The reaction is normally carried out using the catalysts described by Tohda, Y. et al. cited above, namely copper(I)iodide/triphenylphosphine-palladium dichloride. The reaction is suitably carried out in triethylamine, a mixture of triethylamine and pyridine or triethylamine and toluene under a dry inert atmosphere such as nitrogen or argon. The reaction is generally carried out at reduced temperature such as in the range from −80° C. to room temperature, the reaction time typically being from 30 minutes to 6 hours.

In the above reactions, it may be preferred or necessary to protect various sensitive or reactive groups present in the starting materials to prevent said groups from interfering with the reactions. Such protection may be carried out in a well-known manner, e.g. as described in “Protective Groups in Organic Chemistry” by Wuts and Greene, Wiley-Interscience; ISBN: 0471160199; 3nd edition (May 15, 1999). For example, in the reaction between the activated acid derivative and the acetylene derivative, a hydroxy group on Ar¹ and/or Ar² may be protected in the form of the methoxymethyl ether, N,N-dimethylcarbamoyl ester, or allyl ether. The protecting group may be removed after the reaction in a manner known per se.

The ethyne derivative may be prepared by standard methods, e.g. as described by Nielsen, S. F. Et al., Bioorg. Med. Chem. 6, pp 937-945 (1998). The carboxylic acids may likewise be prepared by standard procedures or by reductive amination as described in the examples.

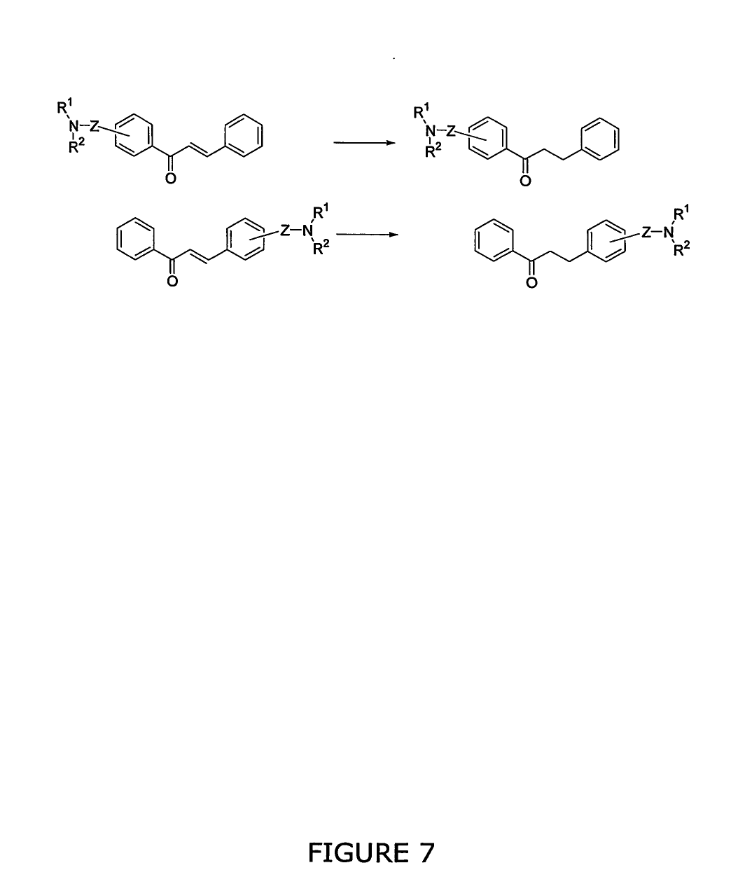

Compounds of the general formula II in which V is —CH₂—CH₂— can be prepared by ionic hydrogenation of the corresponding α,β-unsaturated compound where V is —CH═CH— as it has been described by the inventors in Nielsen, S. F. et al. Tetrahedron, 53, pp 5573-5580 (1997) (see FIG. 7).

Further possible synthetic routes for the preparation of the saturated variants are described in “Advanced Organic Chemistry” by Jerry March, 3rd ed. (especially chapter 15, pages 691-700) and references cited therein. Thus, it is possible to obtain a large variety of compounds of the 1,3-bis-aromatic-propan-1-one type from the corresponding prop-2-en-1-ones.

This being said, the present invention also provides a method for the preparation of the compounds of the general formula I.

### Therapeutic Uses

The present inventor has found that that the novel compound has interesting properties as bacteriocidal agents (see the Examples section). It is of course possible that the compounds also have other interesting properties to be utilised in the medical field.

Thus, the present invention provides, in a further aspect, a compound (a chalcone derivative/analogue) as defined herein for use as a drug substance.

In one aspect, the chalcone derivatives/analogues may be used for the treatment of bacterial infections in a mammal in need thereof. Such bacterial infection may be associated with common Gram-positive and/or Gram-negative pathogenes or with microaerophilic or anaerobic bacteria. As a particularly relevant example of bacteria against which chalcone derivatives/analogues demonstrate an effect can be mentioned antibiotic-sensitive or—resistant strains of S. aureus and/or E. faecium. Other examples include community acquired and nosocomial respiratory infections, including S. pneumoniae, S. pyogenes and members of Enterobactediaceae (e.g. E. coli), microaerophilic bacteria associated with gastric disease (e.g. Helicobacter pylori) or pathogenic anaerobic bacteria (e.g. Bacteroides fragilis and Clostridium species).

Thus, the present invention also provides the use of a compound of the general formula I for the preparation of a pharmaceutical composition for the treatment of bacterial infections, in particular the bacterial infections described above.

The present invention also provides a method for treating bacterial infections (in particular the bacterial infections described above) in a mammal comprising administration of a compound of the general formula I to a subject in need therefor.

It is furthermore envisaged that the compounds of the general formula I can be used in combination with a second antibiotic compound in order to provide a more efficient treatment of bacterial infections as those mentioned above. Thus, pharmaceutical compositions comprising a compound of the general formula I and a second antibiotic compound are also envisaged within the scope of the present invention.

### Formulation of Pharmaceutical Compositions

The chalcone derivatives/analogues are typically formulated in a pharmaceutical composition prior to use as a drug substance.

The administration route of the compounds as defined herein may be any suitable route which leads to a concentration in the blood or tissue corresponding to a therapeutic effective concentration. Thus, e.g., the following administration routes may be applicable although the invention is not limited thereto: the oral route, the parenteral route, the cutaneous route, the nasal route, the rectal route, the vaginal route and the ocular route. It should be clear to a person skilled in the art that the administration route is dependent on the particular compound in question, particularly the choice of administration route depends on the physico-chemical properties of the compound together with the age and weight of the patient and on the particular disease or condition and the severity of the same.

The compounds as defined herein may be contained in any appropriate amount in a pharmaceutical composition, and are generally contained in an amount of about 1-95% by weight of the total weight of the composition. The composition may be presented in a dosage form which is suitable for the oral, parenteral, rectal, cutaneous, nasal, vaginal and/or ocular administration route. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, aerosols and in other suitable form.

The pharmaceutical compositions may be formulated according to conventional pharmaceutical practice, see, e.g., “Remington's Pharmaceutical Sciences” and “Encyclopedia of Pharmaceutical Technology”, edited by Swarbrick, J. & J. C. Boylan, Marcel Dekker, Inc., New York, 1988. Typically, the compounds defined herein are formulated with (at least) a pharmaceutically acceptable carrier or exipient. Pharmaceutically acceptable carriers or exipients are those known by the person skilled in the art.

Thus, the present invention provides in a further aspect a pharmaceutical composition comprising a compound of the general formula I in combination with a pharmaceutically acceptable carrier.

Pharmaceutical compositions according to the present invention may be formulated to release the active compound substantially immediately upon administration or at any substantially predetermined time or time period after administration. The latter type of compositions is generally known as controlled release formulations.

In the present context, the term “controlled release formulation” embraces i) formulations which create a substantially constant concentration of the drug within the body over an extended period of time, ii) formulations which after a predetermined lag time create a substantially constant concentration of the drug within the body over an extended period of time, iii) formulations which sustain drug action during a predetermined time period by maintaining a relatively, constant, effective drug level in the body with concomitant minimization of undesirable side effects associated with fluctuations in the plasma level of the active drug substance (sawtooth kinetic pattern), iv) formulations which attempt to localize drug action by, e.g., spatial placement of a controlled release composition adjacent to or in the diseased tissue or organ, v) formulations which attempt to target drug action by using carriers or chemical derivatives to deliver the drug to a particular target cell type. Controlled release formulations may also be denoted “sustained release”, “prolonged release”, “programmed release”, “time release”, “rate-controlled” and/or “targeted release” formulations.

Controlled release pharmaceutical compositions may be presented in any suitable dosage forms, especially in dosage forms intended for oral, parenteral, cutaneous nasal, rectal, vaginal and/or ocular administration. Examples include single or multiple unit tablet or capsule compositions, oil solutions, suspensions, emulsions, microcapsules, microspheres, nanoparticles, liposomes, delivery devices such as those intended for oral, parenteral, cutaneous, nasal, vaginal or ocular use.

Preparation of solid dosage forms for oral use, controlled release oral dosage forms, fluid liquid compositions, parenteral compositions, controlled release parenteral compositions, rectal compositions, nasal compositions, percutaneous and topical compositions, controlled release percutaneous and topical compositions, and compositions for administration to the eye can be performed essentially as described in the applicant's earlier international application No. WO 99/00114, page 29, line 9, to page 40, line 3. Also, and more generally, the formulation and preparation of the above-mentioned compositions are well-known to those skilled in the art of pharmaceutical formulation. Specific formulations can be found in “Remington's Pharmaceutical Sciences”.

### Dosages

The compound are preferably administered in an amount of about 0.1-50 mg per kg body weight per day, such as about 0.5-25 mg per kg body weight per day.

For compositions adapted for oral administration for systemic use, the dosage is normally 2 mg to 1 g per dose administered 1-4 times daily for 1 week to 12 months depending on the disease to be treated.

The dosage for oral administration for the treatment of bacterial diseases is normally 1 mg to 1 g per dose administered 1-4 times daily for 1 week to 12 months; in particular, the treatment of tuberculosis will normally be carried out for 6-12 months.

The dosage for oral administration of the composition in order to prevent diseases is normally 1 mg to 75 mg per kg body weight per day. The dosage may be administered once or twice daily for a period starting 1 week before the exposure to the disease until 4 weeks after the exposure.

For compositions adapted for rectal use for preventing diseases, a somewhat higher amount of the compound is usually preferred, i.e. from approximately 1 mg to 100 mg per kg body weight per day.

For parenteral administration, a dose of about 0.1 mg to about 50 mg per kg body weight per day is convenient. For intravenous administration, a dose of about 0.1 mg to about 20 mg per kg body weight per day administered for 1 day to 3 months is convenient. For intraarticular administration, a dose of about 0.1 mg to about 20 mg per kg body weight per day is usually preferable. For parenteral administration in general, a solution in an aqueous medium of 0.5-2% or more of the active ingredients may be employed.

For topical administration on the skin, a dose of about 1 mg to about 5 g administered 1-10 times daily for 1 week to 12 months is usually preferable.

In many cases, it will be preferred to administer the compound defined herein together with another antibiotic drug, thereby reducing the risk of development of resistance against the conventional drugs, and reducing the amount of each of the drugs to be administered, thus reducing the risk of side effects caused by the conventional drugs.

### Screening

In a further aspect, the invention further provides combinatorial libraries, mixtures and kits for screening compounds as defined above.

In one embodiment, a combinatorial library comprising at least two compounds of the general formula I is provided. Such library may be in the form of an equimolar mixture, or in a mixture of any stoichiometry. Typical embodiments comprise at least two, such as at least 10, such as at least 100, such as at least 1000, such as at least 10,000, such as at least 100,000 compounds as defined above.

In another embodiment, combinatorial compound collections in the form of kits for screening for biologically or pharmacologically active compounds are provided. Such kits comprise at least two topologically distinct singular compounds of the general formula I. Typical kits comprise at least 10, such as at least 100, such as at least 1000, such as at least 10,000, such as at least 100,000 compounds as defined above. Kits are preferably provided in the form of solutions of the compounds in appropriate solvents.

Further provided are methods for screening for pharmacologically active compounds, especially bacteriocidal agents, consisting of the steps of preparing a kit or library comprising at least two compounds of the general formula I, contacting said kit or library with a target molecule, such as a protein or nucleic acid, a target tissue, or a target organism, such as a bacterium and detecting a biological or pharmacological response caused by at least one compound. Optionally, the steps may be repeated when appropriate to achieve deconvolution.

### EXAMPLES

### Preparation of Compounds

Chemical names presented below were generated using the software ChemDraw Ultra, version 6.0.1, from CambridgeSoft.com.

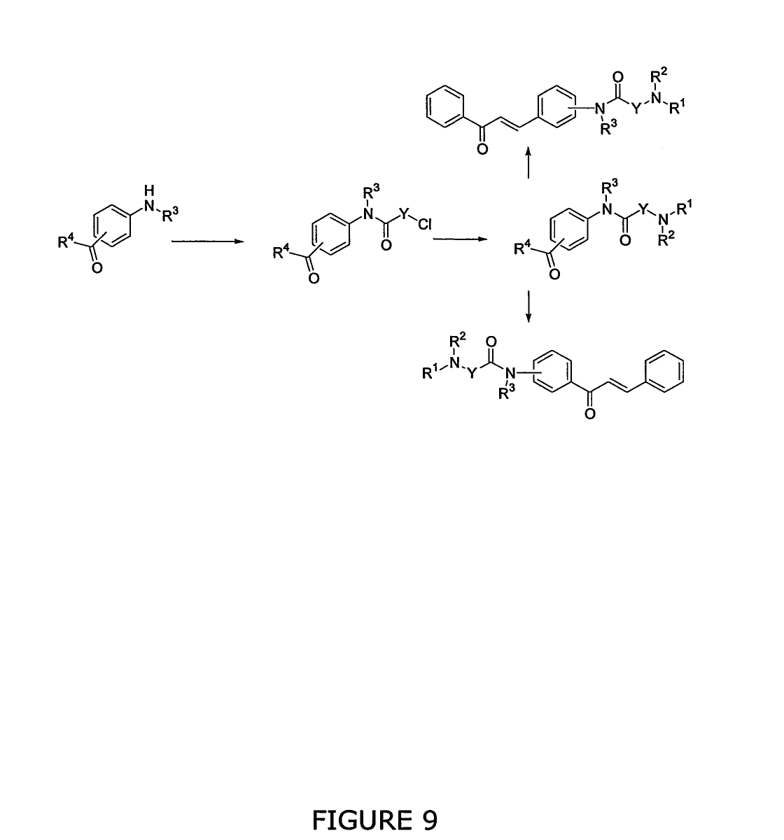

The general method for the preparation of the A ring or B ring having the amino-functional group is illustrated in FIGS. 6, 8, 9 and 11.

### Characterisation of the Compounds

The compounds were characterised by NMR (300 MHz) and GC-MS/LC-MS.

General procedure A—Preparation of alkyl—or dialkyl aminomethyl acetophenones

To a solution of (2-methyl-[1,3]dioxan-2-yl) benzaldehyde (165 mmol) and amine (247 mmol) in dry THF (1.5 L) was added sodium triacetoxyborohydride (257 mmol) under argon. The resulting suspension was stirred at room temperature for 18 hr. A solution of sodium hydroxide (2M) was added and stirring was continued for approximately 30 min, before the mixture was acidified using HCl (6M). The mixture was stirred for 1 hr. and extracted with diethyl ether, which was discarded. The pH of the aqueous phase was adjusted to 11-14 using sodium hydroxide and extracted again with diethyl ether. The latter organic phase, was dried (Na₂SO₄), filtered and evaporated to give the title products, which were used without further purification.

General procedure B—Preparation of (2-dimethylaminoethoxy)-benzaldehydes

A stirred solution of hydroxybenzaldehyde (59.7 mmol) in dry toluene (200 mL) and DMSO (1 mL) was added 60% NaH (60 mmol) under ice cooling. The reaction was slowly heated to room temperature. 2-(dimethylamino)ethyl chloride, HCl (110 mmol) dissolved in water (50 mL) was added NaOH (110 mmol) and the aqueous phase was extracted with toluene (3×30 mL). The combined organic phases were dried (Na₂SO₄) and slowly added to the reaction. The solution was heated to 90° C. for 16 h. The reaction mixture was cooled to room temperature and washed with water (3×100 mL), 2N NaOH (100 mL) and dried (Na₂SO₄). Evaporation in vacuo gave the title products.

General procedure C—Nucleophilic aromatic substitution on Fluorobenzaldehyde

A stirred solution of fluoro-benzaldehyde (49.3 mmol) in dry DMF (50 mL) was added K₂CO₃ (10.2 g, 74.0 mmol) and amine (74 mmol) and left overnight at 100° C. The reaction mixture was cooled to room temperature, added water (100 mL) and extracted with ether (3×50 mL). The combined organic phases was washed with water and dried (Na₂SO₄). Evaporation in vacuo and recrystallization (heptane) gave the product.

General procedure D—Preparation of biaryl carbaldehydes

A solution of Na₂CO₃ (44 mmol) in water (20 mL) was added to a solution of bromobenzaldehyde (14.7 mmol) and (hetero)arylboronic acid (17.6 mmol) in DME (40 mL). The mixture was flushed with argon for 2 minutes followed by addition of Pd(PPh₃)₂Cl₂ (310 mg, 3 mol %). The reaction was heated at reflux and left overnight under an atmosphere of argon. The reaction was cooled, 2M Na₂CO₃ was added, and the mixture was extracted with EtOAc (3×20 mL). The title products were purified by flash chromatography.

General Procedure E—Synthesis of (hetero)aryl chalcones from bromo-chalcones

A solution of bromo-chalcone (0.92 mmol) and (hetero)aryl boronic acid (1.11 mmol) in DME (10 mL) was added a solution of Na₂CO₃ (290 mg, 2.8 mmol) in water (5 mL). The mixture was bubbled through with argon for 2 minutes followed by addition of Pd(PPh₃)₂Cl₂ (19 mg, 3 mol %). The reaction was heated to reflux and left overnight under an atmosphere of argon. The solution was cooled, added 2M Na₂CO₃, extracted with ether (3×20 mL) and purified by column chromatography.

General procedure F—Synthesis of (amino-)chalcones

To a solution of an acetophenone (2 mmol) and a benzaldehyde (2 mmol) in 96% EtOH (10 mL) was added 8M NaOH (0.3 mL), and the mixture was stirred for 3-18 hours at 25° C. The mixture was evaporated on Celite® and the product was isolated by flash chromatography. The amino-functional chalcone was dissolved in MeOH:Et₂O (1:9 v/v, 10 mL) and a solution of fumaric acid or oxalic acid in MeOH:Et₂O (1:9 v/v) was added. The salt was filtered off and recrystallised from MeOH or MeCN. Some-amino-functional chalcones did not undergo salt formation, and was isolated as the free base. The purity was >95% determined by HPLC and the molecular weight was determined by LC-MS.

General procedure G—Synthesis of chalcones with one quaternary ammonia salt.

Iodomethane (1 mmol) was added to a solution of the amine (1 mmol) in THF (10 ml) and stirring was continued for 16 hours. The precipitate was collected by filtration.

General procedure H—Synthesis of chalcones with more than one quaternary ammonia salt.

A solution of the amine (1 mmol) in iodomethane (100 mmol) was vigorously stirred for 16 hours. Iodomethane was removed under reduced pressure and the residue was washed with THF to give the product as crystals.

### Preparation of Starting Materials

SM-001 1-(3-Dimethylaminomethyl-phenyl)-ethanone

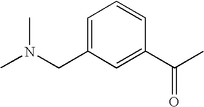

General procedure A gave the title product as yellow oil in 89% yield.

^{H}-NMR (CDCl₃) δ 7.89 (s, 1H), 7.85 (d, 1H), 7.52 (d, 1H), 7.42 (t, 1H), 3.47 (s, 2H), 2.61 (s, 3H), 2.25 (s, 6H).

SM-002 5-Bromo-2-(2-dimethylamino-ethoxy)-benzaldehyde

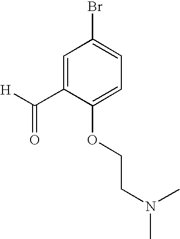

General procedure B gave the title compound as a yellow oil in 65% yield.

1H-NMR (CDCl3) δ 10.43 (s, 1H), 7.94 (d, 1H), 7.63 (dd, 1H), 6.92 (d, 1H), 4.19 (t, 2H), 2.81 (t, 2H), 2.37 (s, 6H).

SM-003 4-(2-Dimethylamino-ethoxy)-biphenyl-3-carbaldehyde

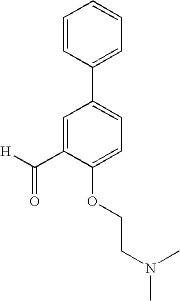

General procedure D gave the title compound as yellow crystals in 57% yield.

1H-NMR (CDCl3) δ 10.48 (s, 1H), 8.01 (d, 1H), 7.71 (dd, 1H), 7.49 (d, 1H), 7.36 (t, 2H), 7.26 (t, 1H), 7.00 (d, 1H), 4.18 (t, 2H), 2.77 (t, 2H), 2.31 (s, 6H).

SM-004 4-(2-Dimethylamino-ethoxy)-3′,5′-dimethyl-biphenyl-3-carbaldehyde

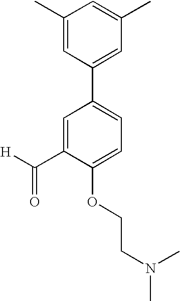

General procedure D gave the title compound as colourless crystals in 81% yield.

¹H-NMR (DMSO-d₆) δ 10.41 (s, 1H), 7.94-7.89 (m, 2H), 7.33 (d, 1H), 7.24 (bs, 2H), 6.98 (bs, 1H), 4.25 (t, 2H), 2.71 (t, 2H), 2.32 (s, 6H), 2.24 (s, 6H).

SM-005 5-Bromo-2-(4-methyl-piperazin-1-yl)-benzaldehyde

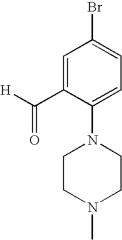

General procedure C gave the title compound as yellow crystals in 62% yield.

¹H NMR (CDCl₃) δ 10.24 (s, 1H), 7.91 (d, 1H), 7.61 (dd, 1H), 7.02 (d, 1H), 3.11 (t, 4H), 2.63 (t, 4H), 2.39 (s, 3H).

SM-006 3-[5-Bromo-2-(4-methyl-piperazin-1-yl)-phenyl]-1-(2-fluoro-4-methoxy-phenyl)-propenone

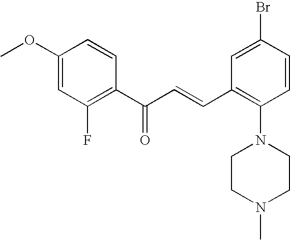

General procedure F gave the title compound as yellow crystals in 65% yield.

¹H NMR (CDCl₃): δ 8.05 (dd, 1H), 7.91 (t, 1H), 7.76 (d, 1H), 7.46 (dd, 1H), 7.41 (dd, 1H), 6.96 (dd, 1H), 6.82 (dd, 1H), 6.69 (dd, 1H), 3.90 (s, 3H), 3.01 (t, 4H), 2.64 (bs, 4H), 2.39 (s, 3H).

SM-007 (E)-1-(2-Chloro-4-methoxy-phenyl)-3-[4-(2-dimethylamino-ethoxy)-3′,5′-dimethyl-biphenyl-3-yl]-propenone

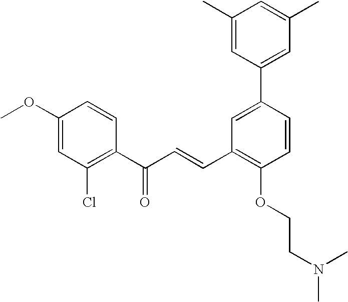

General procedure F gave the fumarate of the title product as yellow crystals in 77% yield.

¹H-NMR (DMSO-d₆) δ 8.05 (d, 1H), 7.76 (d, 1H), 7.71 (dd, 1H), 7.60 (d, 1H), 7.51 (d, 1H), 7.32 (bs, 2H), 7.18 (d, 1H) 7.16 (d, 1H), 7.05 (dd, 1H), 6.96 (bs, 1H), 6.59 (s, 2H), 4.22 (t, 2H), 3.86 (s, 3H), 2.82 (t, 2H), 2.33 (s, 6H), 2.29 (s, 6H).

SM-008 (E)-1-(4-Amino-phenyl)-3-[4-(2-dimethylamino-ethoxy)-3′,5′-dimethyl-biphenyl-3-yl]-propenone

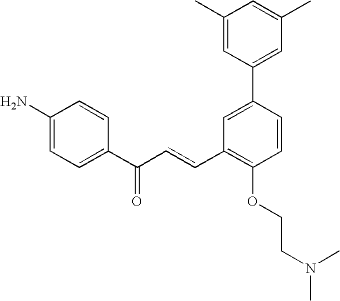

General procedure F gave the fumarate of the title compound as yellow crystals in 20% yield.

¹H-NMR (DMSO-d₆) δ 8.17-8.02 (m, 2H), 8.02-7.90 (m, 3H), 7.68 (dd, 1H), 7.37 (s, 2H), 7.23 (d, 1H), 7.02 (s, 1H), 6.66 (d, 2H), 6.63 (s, 2H), 6.17 (s, 2H), 4.30 (t, 2H), 2.95 (t, 2H), 2.43 (s, 6H), 2.38 (s, 6H).

SM-009 (E)-1-(2-Amino-phenyl)-3-[4-(2-dimethylamino-ethoxy)-3′,5′-dimethyl-biphenyl-3-yl]-propenone

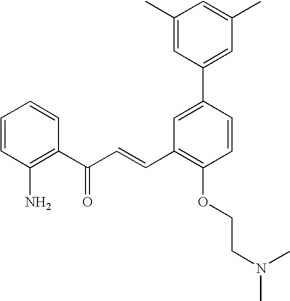

General procedure F gave the fumarate of the title compound as yellow crystals in 53% yield.

¹H-NMR (DMSO-d₆) δ 8.18-8.05 (m, 3H), 7.94 (d, 1H), 7.66 (dd, 1H), 7.45-7.23 (m, 5H), 7.19 (d, 1H), 6.97 (s, 1H), 6.80 (d, 1H), 6.64-6.53 (m, 2H), 4.25 (t, 2H), 2.84 (t, 2), 2.33 (s, 6H).

SM-010 (E)-1-(2-Fluoro-4-methoxy-phenyl)-3-[2′-methoxy-4-(4-methyl-piperazin-1-yl)-biphenyl-3-yl]-propenone

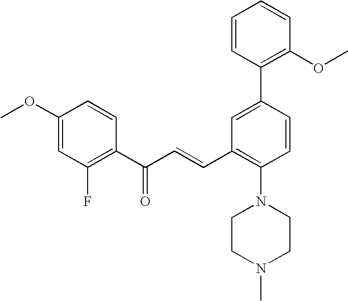

General Procedure E gave the fumarate salt of title compound as yellow crystals in 33% yield.

¹H-NMR (DMSO-d₆): δ 7.76 (dd, 1H), 7.65 (t, 1H), 7.67 (d, 1H), 7.37 (dd, 1H), 7.29 (dd, 1H), 7.20-7.15 (m, 2H), 7.02-6.75 (m, 5H), 6.44 (s, 3H), 3.70 (s, 3H), 3.61 (s, 3H), 2.81 (t, 4H), 2.45 (bs, 4H), 2.17 (s, 3H).

SM-011 (E)-3-(4-Dibutylamino-phenyl)-1-(3-dimethylaminomethyl-phenyl)-propenone

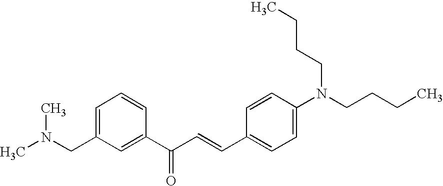

General procedure F gave the title compound as orange oil in 24% yield.

¹H-NMR(CDCl₃): δ 7.91 (d, 2H), 7.84 (d, 1H), 7.75-7.41 (m, 4H), 7.32 (d, 1H), 6.62 (d, 2H), 3.58 (s, 2H), 3.32 (t, 4H), 2.26 (s, 6H), 1.64-1.54 (m, 4H), 1.46-29 (m, 4H), 0.97 (t, 6H).

SM-012 (E)-3-[4-(2-Dimethylamino-ethoxy)-biphenyl-3-yl]-1-(3-dimethylaminomethyl-phenyl)-propenone

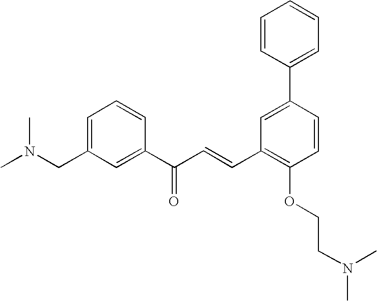

General procedure F gave the title compound as yellow oil in 42% yield.

¹H-NMR (CDCl₃) δ 8.07 (d, 1H), 7.90-7.88 (m, 2H), 7.77 (d, 1H), 7.71 (d, 1H), 7.54-7.47 (m, 4H), 7.41-7.38 (m, 3H), 7.36-7.25 (m, 1H), 6.96 (d, 1H), 4.15 (t, 2H), 3.44 (s, 2H), 2.80 (t, 2H), 2.31 (s, 6H), 2.20 (s, 6H).

SM-013 (E)-3-[4-(2-Dimethylamino-ethoxy)-3′,5′-dimethyl-biphenyl-3-yl]-1-(2-dimethylaminomethyl-4-methoxy-phenyl)-propenone

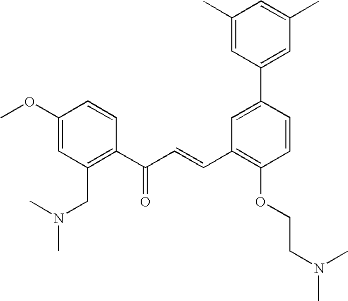

General procedure F gave the title compound as off-white crystals in 48% yield.

¹H-NMR (DMSO-d₆) δ 8.01 (d, 1H), 7.68-7.63 (m, 2H), 7.51 (d, 1H), 7.47 (d, 1H), 7.32 (bs, 2H), 7.15 (d, 1H), 7.04 (d, 1H), 6.96 (bs, 1H), 6.92 (dd, 1H, 4.15 (t, 2H), 3.82 (s, 3H), 3.55 (s, 2H), 2.63 (t, 2H), 2.33 (s, 6H), 2.15 (s, 6H), 2.07 (s, 6H).

### Preparation of the Quaternary Amino-Functional Chalcone Derivatives/Analogues

D-001 (2-{3-[3-(2-Chloro-4-methoxy-phenyl)-3-oxo-propenyl]-3′,5′-dimethyl-biphenyl-4-yloxy}-ethyl)-trimethyl-ammonium; iodide

¹H NMR (d-DMSO): 8.14 (d, 1H), 7.79 (dd, 1H), 7.78 (d, 1H), 7.64 (d, 1H), 7.51 (d, 1H), 7.34 (s, 2H), 7.25 (d, 1H), 7.17 (d, 1H), 7.06 (dd, 1H), 700 (s, 1H), 4.60 (b, 2H), 3.88 (m, 5 H), 3.17 (s, 9H), 2.34 (s., 6H).

Prepared by general procedure G giving the compound as yellow crystals.

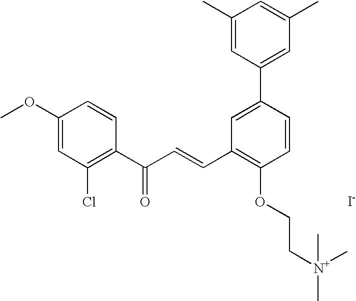

D-002 (2-{3-[3-(4-Amino-phenyl)-3-oxo-propenyl]-3′,5′-dimethyl-biphenyl-4-yloxy}-ethyl)-trimethyl-ammonium; iodide

Prepared by general procedure G giving the compound as yellow crystals.

¹H-NMR (DMSO): 8.06 (d, 1H), 7.8 (m, 4H), 7.55 (dd, 1H) 7.29 (s, 2H), 7.09 (d, 1H), 6.82 (s, 1H), 6.48 (d, 2H), 6.03 (s, 2H), 4.46 (t, 2H, 3.75 (t, 2H), 3.08 (s, 9H), 2.17 (s, 6H).

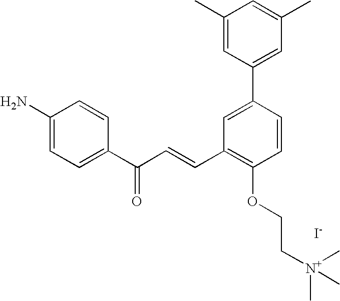

D-003 (2-{3-[3-(2-Amino-phenyl)-3-oxo-propenyl]-3′,5′-dimethyl-biphenyl-4-yloxy}-ethyl)-trimethyl-ammonium; iodide

Prepared by general procedure G giving the compound as yellow crystals.

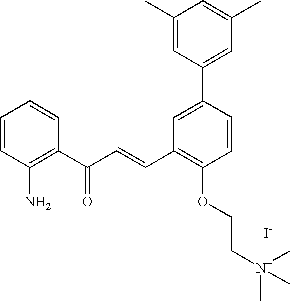

¹H NMR (DMSO): 8.32 (d, 1H), 8.18 (d, 1H), 8.08 (bs, 2H), 7.77 (dd, 1H), 7.5-7.3 (m, 6H), 7.07 (s, 1H), 6.86 (d, 1H), 6.66 (t, 1H), 4.67 (b, 2H), 3.98 (bt 2H), 3.30 (s, 9H), 2.42 (s, 6H).

D-004 4-{3-[3-(2-Fluoro-4-methoxy-phenyl)-3-oxo-propenyl]-2′-methoxy-biphenyl-4-yl}-1,1-dimethyl-piperazin-1-ium

Prepared by general procedure G (0.05 mmol scale) giving the product as yellow crystals (56%, 0.02 g). LC-MS: 475.2 (M+). ¹H NMR (DMSO-d6): δ 7.81 (1H, d J=16.8 Hz), 7.71-7.60 (2H, m), 7.48 (1H, dd J=8.4 Hz, 1.8 Hz), 7.32-7.17 (4H, m), 7.04 (1H, d J=7.5 Hz), 6.96 (1H, J=7.5 Hz), 6.79 (1H, dd J=9.0 Hz, 1.9 Hz), 6.72 (1H, dd J=13.0 Hz, 2.2 Hz), 3.73 (3H, s), 3.64 (3H, s), 3.48 (4H, m), 3.18 (4H, m), 3.10 (6H, s). blood agar plates. MBC was determined as the minimal concentration of test compound able to decrease colony count on these plates below 0.1% compared to the original inoculum.

### Killing Curve Determination

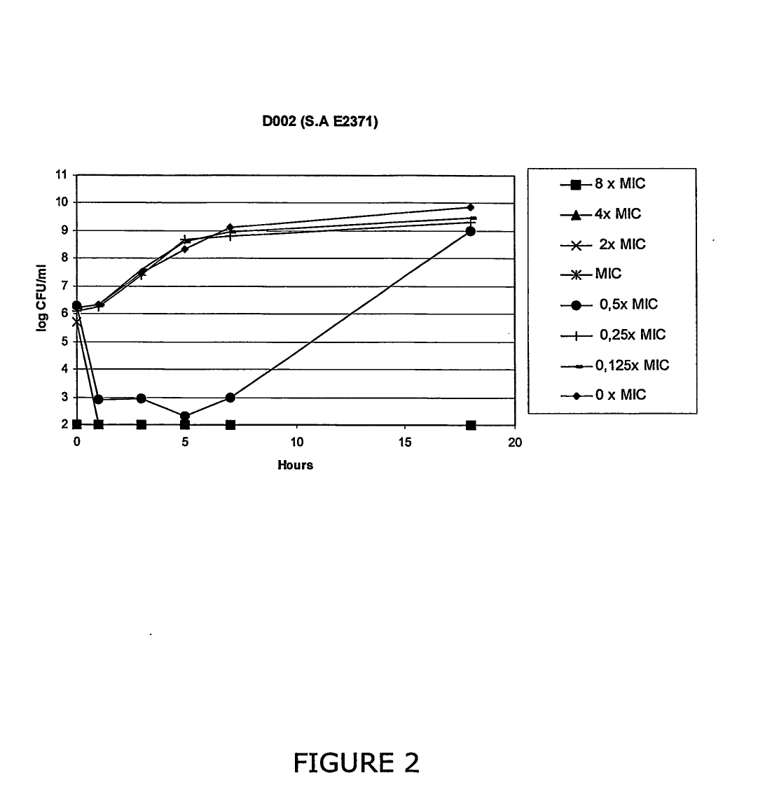

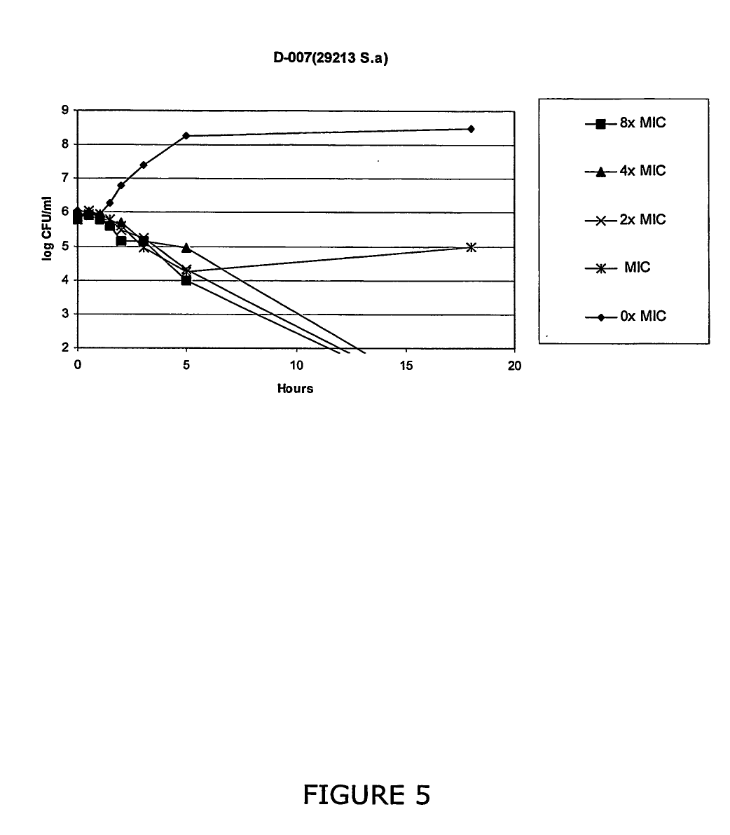

For the determination of the killing curve of a test compound a dilution series of test compound was made and inoculated with approximately 5×10E5 CFU/ml as described for the MIC macrodilution assay above. At the timepoints indicated 100 μl samples was withdrawn from the test tubes, serially diluted and spotted in duplicate on unselective agar plates to determine CFU. Test compounds with bactericidal activity is capable of decreasing surviving colony counts (CFU/ml) when incubated with bacteria. Bactericidal activity may be either primarily dependent on concentration of test compound or on incubation time with test compound. An example of a bactericidal compound (D-003), which is primarily dependent on the concentration of the test compound is shown in FIG. 2. An example of a bactericidal compound (D-007) which is primarily dependent on the incubation time with the compound is shown in FIG. 5.

### MIC Determination Against Helicobacter pylori

Six strains of Helicobacter pylori were used in an agar dilution assay according to the standards of NCCLS (National Committee for Clinical Laboratory Standards. Methods for Dilution Antimicrobial Susceptibility Tests for Bacteria That Grow Aerobically; Approved Standard—Fifth Edition. M7-A5 NCCLS 2000). MH-agar plates supplemented with 5% horse blood and containing a dilution series of the test compound were inoculated in duplicate with 10 μl spots of a 2 McF suspension of the different strains of H. pylori. This inoculum corresponds to approximately 10E6 CFU/spot. Plates were then incubated in a microaerophlic atmosphere at 35° C. for 72 hours. The MIC endpoint was determined as the lowest concentration of test compound able to completely inhibit or most significantly reduce growth compared to growth control plates not containing test compounds.

### Activity Determination Against Anaerobic Bacteria

Screening for activity against anaerobic bacteria was done against two isolates of Bacteroides fragilis, an isolate of Clostridium difficile and an isolate of Clostridium perfringens in an agar dilution assay as described by NCCLS (National Committee for Clinical Laboratory Standards. Methods for Antimicrobial Susceptibility Testing of Anaerobic Bacteria; Approved Standard—Fifth Edition. M11-A5 NCCLS 2000) with the exception that Mueller-Hinton agar was used in place of supplemented Brucella broth. Plates containing test compound at a single concentration (either 100 or 150 μM) were prepared in duplicate along with appropriate

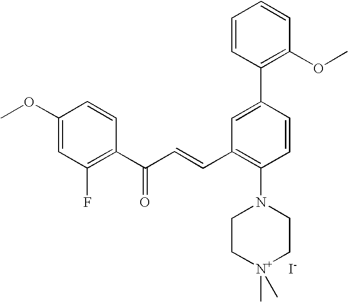

D-005 {3-[3-(4-Dibutylamino-phenyl)-acryloyl]-benzyl}-trimethyl-ammonium

Prepared by general procedure G (0.17 mmol scale) giving the product as red gum (48%, 0.044 g).

LC-MS: 407.3 (M+). ¹H NMR (DMSO-d6): δ 8.28-8.21 (2H, m), 7.80-7.66 (5H, m), 7.58 (1H, d J=15.2 Hz), 6.78 (2H, d J=7.5 Hz), 4.65 (2H, s), 3.35 (4H, m), 3.08 (9H, s), 1.53 (4H, p J=7.4 Hz), 1.35 (4H, six. J=7.4 Hz), 0.93 (6H, t J=7.4 Hz).

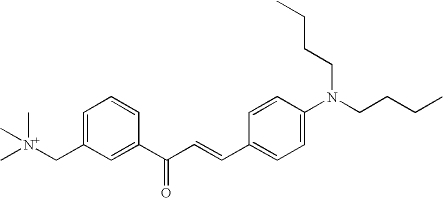

D-006 (E)-3-[4-(2-Trimethylammonium-ethoxy)-biphenyl-3-yl]-1-(3-trimethylammonium-phenyl)-propenone, di-iodide.

Prepared by general procedure H (0.16 mmol scale) giving the product as yellow crystals (66%, 0.07 g). LC-MS: 458.3 (M+). ¹H NMR (DMSO-d6): δ 8.43 (1H, d J=6.5 Hz), 8.24 (1H, d J=2.1 Hz), 8.25 (1H, bs), 8.14 (1H, d J=15.5 Hz), 8.07 (1H, d J=15.5 Hz), 7.90-7-73 (5H, m), 7.50 (2H, t J=7.6 Hz), 7.42-7.30 (2H, m), 4.67 (4H, bs), 3.94 (2H, t J=4.4 Hz), 3.26 (9H, s), 3.08 (9H, s).

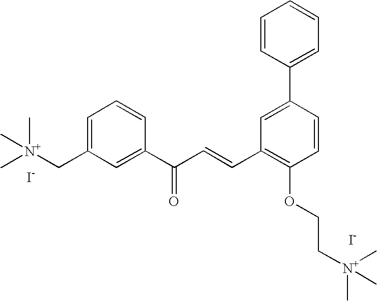

D-007 (E)-3-[4-(2-trimethylammonium-ethoxy)-3′,5′-dimethyl-biphenyl-3-yl]-1-(2-trimethylammonium-4-methoxy-phenyl)-propenone, di-iodide

Prepared by general procedure H (0.2 mmol scale) giving the product as yellow crystals (76%, 0.12 g). LC-MS: 516.3 (M+). ¹H NMR (DMSO-d6): δ 8.17 (1H, d J 2.3 Hz), 8.13 (1H, d J=8.7 Hz), 7.97 (1H, d J=15.7 Hz), 7.82-7.70 (2H, m), 7.36-7.26 (5H, m), 7.00 (1H, bs), 4.90 (2H, s), 4.63 (2H, bs), 3.93 (3H, s), 3.91 (2H, m), 3.22 (9H, s), 3.04 (9H, s), 2.35 (6H, s).

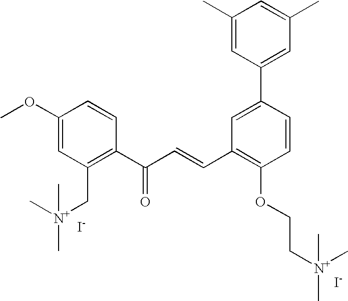

### Pharmacokinetic Studies

Evaluation of the pharmacokinetic properties of the compounds was done using female NMRI mice (weighing app. 30 g). Test articles were administrated intravenously and Samples of serum were taken at defined time-points.

Standards and QC-samples in plasma were prepared and the serum concentrations of the test compounds quantified by HPLC-MS. Prior to analysis, proteins were precipitated by deluding the samples (1:1) (v/v) with 100% acetonitrile followed by centrifugation at 14,000 rpm in 10 min. The supernatant was used for the analysis.

### HPLC-MS System:

A Waters Alliance HPLC-system (Milford, Mass., USA) was coupled to a Quatro Micro triple quadropl mass spectrometer (Micromass, Manchester, UK) operating in positive (ESI) mode. Separation was performed on a XTerra MS C₁₈ column (150*2.1 mm I.D., 3,5 μm particle size) (Waters Milford, Mass., USA). Mobile phase A: 0.1% (v/v) formic acid or 10 mM ammonium acetate pH-adjusted to 9.5 in MilliQ-water, mobile phase B: 100% methanol. The gradient was as follows: 0 min=70% A-30% B, 0-10 min. a linear gradient to 10% A and 90% B this was maintained till 11 min, 11-13 linear gradient to 70% A and 30% B this was maintained till 18 min. The flow rate was 0.20 ml/min, injection volume 10 μl.

Biological Testing

### In Vitro Microbiological Testing

### MIC Determination In Broth Microdilution Assay

Compounds were screened for activity against a panel of 10 different non-fastidious bacteria growing aerobically (Staphylococcus aureus ATCC29213; Staphylococcus aureus ATCC33591; Staphylococcus intermedius #2357 (clinical isolate from the Copenhagen area); Enterococcus faecalis ATCC29212; Enterococcus faecium #17501 (vancomycin-resistant clinical isolate); Streptococcus pneumoniae #998 (clinical isolate); Streptococcus pyogenes #14813 (clinical isolate); Streptococcus agalactiae #19855 (clinical isolate); Eschericia coli ATCC25922 and Eschericia coli ESS). The screening assay was done in 200 μl MH-broth cultures in microtitre plates. For compounds exhibiting activity in the initial screen MIC was determined in a microdilution assay using MH-broth as described by NCLLS (National Committee for Clinical Laboratory Standards. Methods for Dilution Antimicrobial Susceptibility Tests for Bacteria That Grow Aerobically; Approved Standard—Fifth Edition. M7-A5 NCCLS 2000) modified to include uninoculated dilution series of test compounds to facilitate MIC determination if the test compound should precipitate. MIC was determined as the lowest concentration of test compound able to inhibit visible growth of bacteria. MICs for ATCC type strains fell within the limits posted by the NCCLS (National Committee for Clinical Laboratory Standards. Performance Stadards for Antimicrobial Susceptibility Testing; Eleventh Informational Supplement. M100-S11 NCCLS 2001) when tested against vancomycin, tetracycline, gentamycin.

### MIC and MBC Determination in Broth Macrodilution Assay

MIC and MBC of test compounds were determined in a broth macrodilution assay using 2 ml MH-broth cultures and an inoculum of approximately 5×10E5 CFU/ml as described by Amsterdam (Amsterdam, D. Susceptibility testing of antimicrobials in liquid media. In V. Lorian (ed.): Antibiotics in Laboratory Medicin 4. edition. Williams & Wilkins 1996). MIC was determined as the minimal concentration of test compound able to inhibit visible growth of bacteria. Samples from cultures inhibited by test compound were plated onto unselective control plates. Activity was present if growth in the presence of test substance was absent or most significantly reduced compared to growth control plates not containg test compound.

Biological Results

Licochalcone A (LicA) and 4′methoxy chalcone (4′MC) described in WO 93/17671 were used as reference compounds in the following discussion.

### Activity Against Non-Fastidious Bacteria

Licochalcone A exhibit moderate bactericidal activity against common pathogenic Gram-positive non-fastidious bacteria including Staphylococcus aureus, Enterococcus faecalis, Enterococcus faecium, Streptococcus pneumoniae, Streptococcus pyogenes, and Streptococcus agalactiae. Licochalcone A maintains its activity also against antibiotic resistant bacteria, e.g. Staphylococcus aureus ATCC33591 (resistant to methicillin) and Enterococcus faecium #17051 (resistant to vancomycin). In contrast, Licochalcone A have only modest or no activity against the prototype pathogenic Gram-negative bacterium, Eschericia coli. 4′MC as a representative of non-hydroxyl chalcones exhibit no antibacterial effect at all.

In comparison with Licochalcone A, quaternary-aminofunctional-chalcone derivatives/analogues retain the activity of Licochalcone A against pathogenic Gram-positive bacteria including antibiotic-resistant strains (cf. Table 1). The quaternary-aminofunctional-chalcone derivatives/analogues exhibit increased potency against Gram-positive pathogens (e.g. D-001, D-005). In contrast to Licochalcone A, quaternary-aminofunctional-chalcone derivatives/analogues exhibit activity against Eschericia coli. Thus, several quaternary-aminofunctional-chalcone derivatives/analogues, e.g. D-003, exhibit high activity against E. coli ATCC2592 (cf. Table 1). This indicates the potential use of quaternary-aminofunctional-chalcone derivatives/analgoues in the treatment of infections with Gram-negative bacteria.

The quaternary amino-functional chalcone derivatives/analogues retain the activity of parent amino-chalcone against pathogenic Gram-positive and Gram-negative bacteria including antibiotic-resistant strains (cf. Table 1).

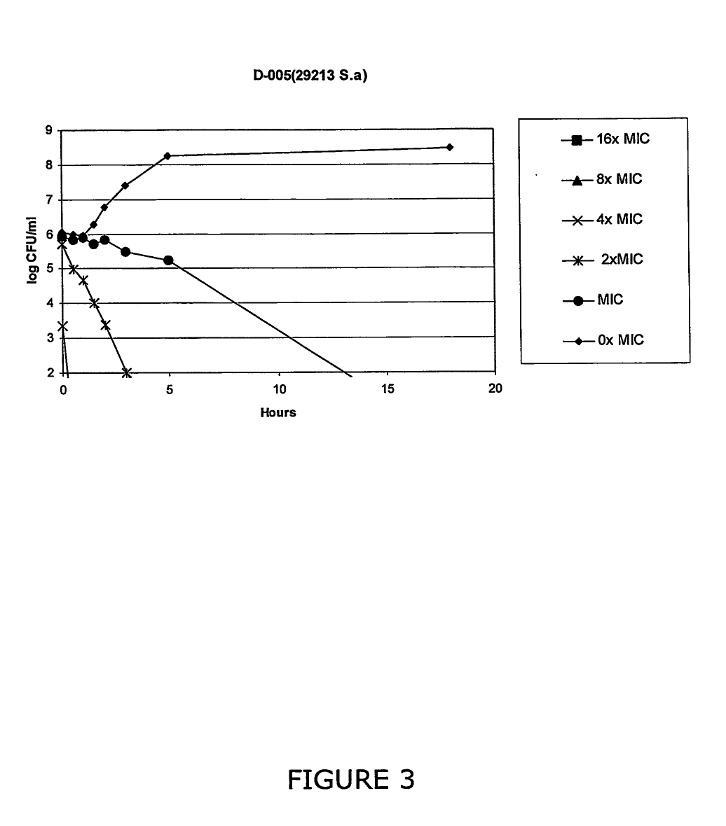

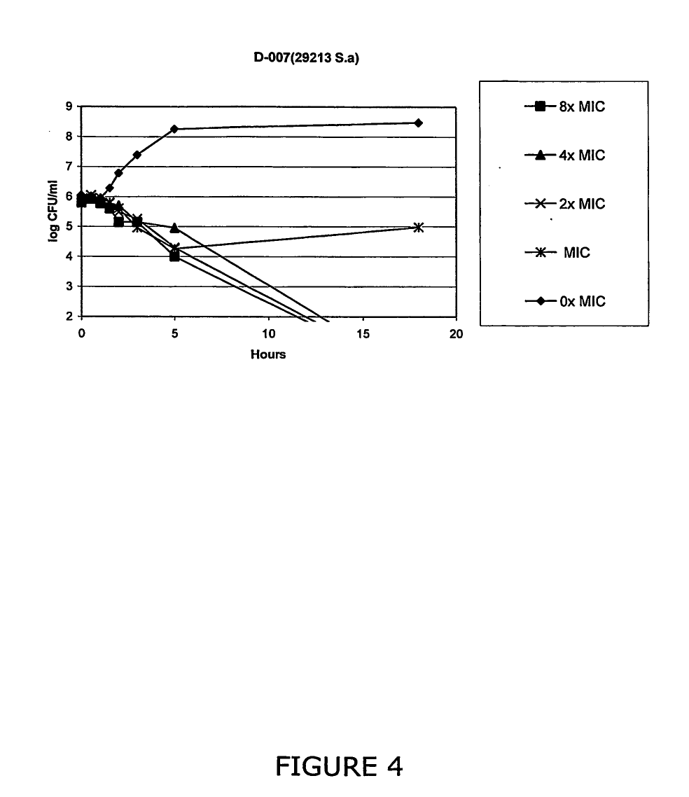

In the treatment of severe infections in immuno-compromised patients, bactericidal action of a antibiotic is a necessity. As exemplified in FIGS. 2, 3, 4 and 5, quaternary-aminofunctional-chalcone derivatives/analogues retain the bactericidal action of Licochalcone A. For some quaternary-aminofunctional-chalcone derivatives/analogues, the bactericidal action is predominantly dependent on the concentration of the compound (e.g. D-003; cf. FIG. 2); for others the bactericidal action is predominantly dependent on the time of incubation with the compound (e.g. D-007; cf. FIG. 5). This knowledge is helpful when designing dosing regimens for in vivo efficacy trials.

TABLE 1
Comparasion of the effect of quaternary amino-chalcone
derivatives/analogues; MIC values in μM.

| | A | B | C | D | E | F | G |
|---|---|---|---|---|---|---|---|
| LicA | 37.5 | 37.5 | 37.5 | 37.5 | 37.5 | 75 | NA |
| 4′-MC | NA | NA | NA | NA | NA | NA | NA |
| D-001 | 2.4 | 4.7 | 2.4 | 4.7 | 4.7 | 18.8 | 37.5 |
| SM007 | 4.7 | 4.7 | 4.7 | 9.4 | 4.7 | 18.8 | NT |
| D-002 | 9.4 | 9.4 | 9.4 | 18.8 | 18.8 | 18.8 | 75 |
| SM008 | 9.4 | 9.4 | 9.4 | 18.8 | 9.4 | 18.8 | NT |
| D-003 | 4.7 | 4.7 | 2.4 | 4.7 | 4.7 | 18.8 | 18.8 |
| SM009 | 4.7 | 4.7 | 4.7 | 9.4 | 4.7 | 18.8 | NT |
| D-004 | 18.8 | 9.4 | 9.4 | 18.8 | 18.8 | 37.5 | 75 |
| SM010 | 4.7 | 9.4 | 9.4 | 18.8 | 9.4 | 37.5 | NT |
| D-005 | 4.7 | 9.4 | 4.7 | 18.8 | 9.4 | 37.5 | 75 |
| SM011 | 9.4 | 9.4 | 9.4 | 9.4 | 9.4 | 18.8 | NA |
| D-006 | 18.8 | 18.8 | 18.8 | 75 | 37.5 | 150 | NA |
| SM012 | 18.8 | 18.8 | 18.8 | 18.8 | 18.8 | 18.8 | 37.5 |
| D-007 | 18.8 | 18.8 | 9.4 | 150 | 75 | 75 | 75 |
| SM013 | 4.7 | 9.4 | 18.8 | 4.7 | 4.7 | 4.7 | 75 |

A: Staphylococcus aureus ATCC29213;
B: Staphylococcus aureus ATCC33591 (resistant to methicillin);
C: Staphylococcus intermedius #2357(clinical Isolate from the Copenhagen area);
D: Enterococcus faecalis ATCC29212;
E: Enterococcus faecium #17501 (vancomycin-resistant clinical isolate);
F: Streptococcus pneumoniae #998 (clinical isolate) and
G: Eschericia coli ATCC25922.
NA: no activity;
NT: not tested.

### Activity Against Helicobacter pylori

Colonization of the gastric mucosa with Helicobacter pylori is an important pathogenic determinant for the development of gastritis and peptic ulcer. The quaternary amino-functional chalcone derivatives/analogues D-001 has been tested for activity against Helicobacter pylori. The compound exhibits MICs in the range between 50 μM and 100 μM when tested against a panel of six strains Helicobacter pylori, that includes strains resistant to metronidazole. Metronidazol is an antibiotic commonly included in treatment regimens designed to eradicate Helicobacter colonization for the treatment of peptic ulcer. The activity of quaternary amino-functional chalcone derivatives/analogues against both metronidazole-resistant and sensitive Helicobacter pylori clearly indicates the potential use of these compounds in the treatment of Helicobacter infections.

### Activity Against Anaerobic Bacteria

The quaternary amino-functional chalcone derivatives/analogues D-001 have been assayed in a single concentration of compound (100 μM) for activity against a panel of anaerobic bacteria containing common human pathogenic bacteria (Bacteroides fragilis, Clostridium perfringens, Clostridium difficele). The compound D-001 exhibit activity against all microorganisms within the test panel. This clearly indicates the potential use of quaternary amino-functional chalcone derivatives/analogues in treatment of infection caused by anaerobic bacteria.

### Pharmacokinetic Profile

The quaternary amino-functional chalcone derivative/analogue D-001 was injected i.v. to mice and the volume of distribution was very low as indicated by the plasma concentration. A single i.v. dose of 5 mg/ml gave a Cₘₐₓ of 126 μg/ml. As the MIC of D-001 is approximately 1 μg/mi very small doses are needed for treatment.

1-38. (canceled)

39. A compound of the general formula I
(Y¹)ₘ—Ar¹)X¹)—C(=O)VAr²(X²)−(Y²)ₚ I
wherein
V designates —CH₂—CH₂—, —CH═CH— or —C≡C—;
Ar¹ and Ar² independently are selected from aryl and heteroaryl;
m is an integer selected from the group consisting of 0, 1, and 2,
p is an integer selected from the group consisting of 0, 1, and 2,
wherein the sum of m and p is at least 1;
each Y¹ and Y² independently represents a substituent selected from A, B, and C
-Z—N⁺(R¹)(R²)R⁴Q⁻, (A)
—NR³-Z—N⁺(R¹)(R²)R⁴Q⁻, and (B)
—O-Z—N⁺(R¹)(R²)R⁴Q⁻; (C)
wherein Z is a biradical —(C(R^{H})₂)ₙ—, wherein n is an integer in the range of 1-6 and each R^{H} is independently selected from hydrogen and C₁₋₆-alkyl, or wherein (R^{H})₂ is =O;
R¹, R² and R⁴ independently are selected from optionally substituted C₁₋₁₂-alkyl, optionally substituted C₂₋₁₂-alkenyl, optionally substituted C₄₋₁₂-alkadienyl, optionally substituted C₆₋₁₂-alkatrienyl, optionally substituted C₂₋₁₂-alkynyl, optionally substituted C₁₋₁₂-alkoxycarbonyl, optionally substituted C₁₋₁₂-alkylcarbonyl, optionally substituted aryl, optionally substituted aryloxycarbonyl, optionally substituted arylcarbonyl, optionally substituted heteroaryl, optionally substituted heteroaryloxycarbonyl, optionally substituted heteroarylcarbonyl, aminocarbonyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl; or R¹ and R² together with the nitrogen atom to which they are attached (—N(R¹)R²) form an optionally substituted nitrogen-containing heterocyclic ring;
R³ is selected from hydrogen, C₁₋₆-alkyl, and C₁₋₆-alkylcarbonyl, said alkyl and alkylcarbonyl optionally carrying substituent(s) selected from halogen, hydroxy, C₁₋₆-alkoxy, carboxy, C₁₋₆-alkoxycarbonyl, C₁₋₆-alkylcarbonyl, amino, mono- and di(C₁₋₆-alkyl)amino, and aryl optionally substituted 1-3 times with C₁₋₄-alkyl, C₁₋₄-alkoxy, nitro, cyano, amino or halogen; or R¹ and R³ together form a biradical Z^{*} which is as defined for Z;
Q is an anion;
X¹ and X² independently designate a substituent present 0-5 times on Ar¹ and Ar², respectively, each X¹ and X² independently being selected from the group consisting of optionally substituted C₁₋₁₂-alkyl, optionally substituted C₂₋₁₂-alkenyl, optionally substituted C₄₋₁₂-alkadienyl, optionally substituted C₆₋₁₂-alkatrienyl, optionally substituted C₂₋₁₂-alkynyl, hydroxy, optionally substituted C₁₋₁₂-alkoxy, optionally substituted C₂₋₁₂-alkenyloxy, carboxy, optionally substituted C₁₋₁₂-alkoxycarbonyl, optionally substituted C₁₋₁₂-alkylcarbonyl, formyl, C₁₋₆-alkylsulphonylamino, optionally substituted aryl, optionally substituted aryloxycarbonyl, optionally substituted aryloxy, optionally substituted arylcarbonyl, optionally substituted arylamino, arylsulphonylamino, optionally substituted heteroaryl, optionally substituted heteroaryloxycarbonyl, optionally substituted heteroaryloxy, optionally substituted heteroarylcarbonyl, optionally substituted heteroarylamino, heteroarylsulphonylamino, optionally substituted heterocyclyl, optionally substituted heterocyclyloxycarbonyl, optionally substituted heterocyclyloxy, optionally substituted heterocyclylcarbonyl, optionally substituted heterocyclylamino, heterocyclylsulphonylamino, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, amino-C₁₋₆-alkyl-carbonylamino, mono- and di(C₁₋₆-alkyl)amino-C₁₋₆-alkyl-carbonylamino, cyano, guanidino, carbamido, C₁₋₆-alkanoyloxy, C₁₋₆-alkylsulphonyl, C₁₋₆-alkylsulphinyl, C₁₋₆-alkylsulphonyloxy, aminosulfonyl, mono- and di(C₁₋₆-alkyl)aminosulfonyl, nitro, optionally substituted C₁₋₆-alkylthio, and halogen, where any nitrogen-bound C₁₋₆-alkyl is optionally substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino;
and salts thereof

40. The compound according to claim 39, wherein R¹, R² and R⁴ independently are selected from optionally substituted C₁₋₁₂-alkyl, optionally substituted C₂₋₁₂-alkenyl, optionally substituted C₂₋₁₂-alkynyl, optionally substituted C₁₋₁₂-alkylcarbonyl, arylcarbonyl, heteroarylcarbonyl, aminocarbonyl, mono- and di(C₁₋₆-alkyl)aminocarbonyl, amino-C₁₋₆-alkyl-aminocarbonyl, and mono- and di(C₁₋₆-alkyl)amino-C₁₋₆-alkyl-aminocarbonyl.

41. The compound according to claim 39, wherein R³ is selected from hydrogen and methyl.

42. The compound according to claim 39, wherein X¹ and X² independently designates 0-4 substituents, where such optional substituents independently are selected from optionally substituted C₁₋₁₂-alkyl, hydroxy, optionally substituted C₁₋₁₂-alkoxy, optionally substituted C₂₋₁₂-alkenyloxy, carboxy, optionally substituted C₁₋₁₂-alkylcarbonyl, formyl, C₁₋₆-alkylsulphonylamino, optionally substituted aryl, optionally substituted aryloxycarbonyl, optionally substituted aryloxy, optionally substituted arylcarbonyl, optionally substituted arylamino, arylsulphonylamino, optionally substituted heteroaryl, optionally substituted heteroarylamino, optionally substituted heteroarylcarbonyl, optionally substituted heteroaryloxy, heteroarylsulphonylamino, optionally substituted heterocyclyl, optionally substituted heterocyclyloxy, optionally substituted heterocyclylamino, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, amino-C₁₋₆-alkyl-carbonylamino, mono- and di(C₁₋₆-alkyl)amino-C₁₋₆-alkyl-carbonylamino, guanidino, carbamido, C₁₋₆-alkylsulphonyl, C₁₋₆-alkylsulphinyl, C₁₋₆-alkylsulphonyloxy, optionally substituted C₁₋₆-alkylthio, aminosulfonyl, mono- and di(C₁₋₆-alkyl)aminosulfonyl, and halogen, where any nitrogen-bound C₁₋₆-alkyl may be substituted with a substituent selected from the group consisting of hydroxy, C₁₋₆-alkoxy, and halogen.

43. The compound according to claim 39, wherein R¹, R² and R⁴ independently are selected from optionally substituted C₁₋₆-alkyl, optionally substituted C₁₋₆-alkylcarbonyl, heteroarylcarbonyl, aminocarbonyl, mono- and di(C₁₋₆-alkyl)aminocarbonyl, amino-C₁₋₆-alkyl-aminocarbonyl, and mono- and di(C₁₋₆-alkyl)amino-C₁₋₆-alkyl-aminocarbonyl.

44. The compound according to claim 39, wherein X¹ and X² independently designate 0-3 substituents, such optional substituents independently being selected from optionally substituted C₁₋₆-alkyl, hydroxy, optionally substituted C₁₋₆-alkoxy, carboxy, optionally substituted C₁₋₆-alkylcarbonyl, C₁₋₆-alkylsulphonylamino, optionally substituted aryl, optionally substituted aryloxy, optionally substituted arylamino, arylsulphonylamino, optionally substituted heteroaryl, optionally substituted heteroarylamino, heteroarylsulphonylamino, amino, mono- and di(C₁₋₆-alkyl)amino, carbamoyl, C₁₋₆-alkylcarbonylamino, guanidino, carbamido, optionally substituted C₁₋₆-alkylthio, optionally substituted heterocyclyl, optionally substituted heterocyclyloxy, optionally substituted heterocyclylamino and halogen, where any nitrogen-bound C₁₋₆-alkyl may be substituted with a substituent selected from the group consisting of hydroxy, C₁₋₆-alkoxy, and halogen.

45. The compound according to claim 39, wherein V designates —CH═CH—.

46. The compound according to claim 39, wherein at least one of Ar¹ and Ar² is phenyl.

47. The compound according to claim 46, wherein both of Ar¹ and Ar² are phenyl, m is 1 or 2, and p is 0, 1 or 2.

48. The compound according to claim 39, wherein X² represents at least one substituent selected from C₁₋₆-alkyl, C₁₋₆-alkoxy, C₁₋₆-alkylcarbonyl, optionally substituted aryl, optionally substituted aryloxy, optionally substituted arylamino, optionally substituted heteroaryl, optionally substituted heteroarylamino, mono- and di(C₁₋₆-alkyl)amino, C₁₋₆-alkyl-carbonylamino, optionally substituted C₁₋₆-alkylthio, optionally substituted heterocyclyl, optionally substituted heterocyclyloxy, optionally substituted heterocyclylamino and halogen.

49. The compound according to claim 39, wherein X² represents at least two halogen atoms.

50. The compound according to claim 39, wherein at least one of Ar¹ and Ar² is selected from the group consisting of thiazolyl, pyrrolyl, imidazolyl, pyrazolyl, pyridinyl, pyrimidinyl, pyrazinyl, pyridazinyl, thienyl, quinolyl, isoquinolyl, and indolyl.

51. The compound according to claim 39, wherein Z is —(CH₂)ₙ—, wherein n is 1-4.

52. The compound according to claim 39, wherein one of Y¹ and Y² represents a substituent of the formula A
—CH₂—N⁺(R¹)(R²)R⁴Q⁻ (A)
wherein R¹, R² and R⁴ are independently C₁₋₆-alkyl.

53. The compound according to claim 51, wherein Y¹ represents a substituent of the formula —CH₂—N⁺(R¹)(R²)R⁴Q⁻.

54. The compound according to claim 39, wherein one of Y¹ and Y² represents a substituent of the formula B
—NR³—(CH₂)₂₋₃—N⁺(R¹)(R²)R⁴Q⁻ (B)
wherein R³ is selected from hydrogen and methyl, and R¹, R² and R⁴ are independently C₁₋₆-alkyl.

55. The compound according to claim 39, wherein one of Y¹ and Y² represents a substituent of the formula C
—O—(CH₂)₂₋₃—N⁺(R¹)(R²)R⁴Q⁻ (C)
wherein R¹, R² and R⁴ are independently C₁₋₆-alkyl.

56. The compound according to claim 51, wherein V is —CH═CH—, and Ar¹ and Ar² both are phenyl.

57. The compound according to claim 39, which is selected from the group consisting of:
(2-{3-[3-(2-Chloro-4-methoxy-phenyl)-3-oxo-propenyl]-3′,5′-dimethyl-biphenyl-4-yloxy}-ethyl)-trimethyl-ammonium, iodide;
(2-{3-[3-(4-Amino-phenyl)-3-oxo-propenyl]-3′,5′-dimethyl-biphenyl-4-yloxy}-ethyl)-trimethyl-ammonium, iodide;
(2-{3-[3-(2-Amino-phenyl)-3-oxo-propenyl]-3′,5′-dimethyl-biphenyl-4-yloxy}-ethyl)-trimethyl-ammonium, iodide;
4-{3-[3-(2-Fluoro-4-methoxy-phenyl)-3-oxo-propenyl]-2′-methoxy-biphenyl-4-yl }-1,1-dimethyl-piperazin-1-ium, iodide;
{3-[3-(4-Dibutylamino-phenyl)-acryloyl]-benzyl}-trimethyl-ammonium, iodide;
3-[4-(2-Trimethylammonium-ethoxy)-biphenyl-3-yl]-1-(3-trimethylammonium-phenyl)-propenone, di-iodide; and
3-[4-(2-trimethylammonium-ethoxy)-3′,5′-dimethyl-biphenyl-3-yl]-1-(2-trimethylammonium-4-methoxy-phenyl)-propenone, di-iodide.

58. A method for treating bacterial infections in a mammal comprising administration of a compound as defined in claim 39.
